(19) United States
(12) Patent Application Publication
(10) Pub. No.: US 2019/0183561 A1
(43) Pub. Date: Jun. 20, 2019

(54) IN SITU THERAPEUTIC CANCER VACCINE CREATION SYSTEM AND METHOD

(71) Applicant: ImmunSYS, Inc., Fort Lauderdale, FL (US)

(72) Inventors: Eamonn Hobbs, Fort Lauderdale, FL (US); Gary M. Onik, Fort Lauderdale, FL (US); James A. Miessau, Branford, CT (US); Jon H. Condra, Doylestown, PA (US)

(21) Appl. No.: 16/222,572

(22) Filed: Dec. 17, 2018

Related U.S. Application Data
(63) Continuation-in-part of application No. 14/451,333, filed on Aug. 4, 2014, now Pat. No. 10,154,869.
Application No. 14/451,333 is a continuation-in-part of application No. 15/102,120, filed on Jun. 6, 2016.
Application No. 15/102,120 is a continuation-in-part of application No. 16/070,072, filed on Jul. 13, 2018.
(60) Provisional application No. 61/867,048, filed on Aug. 17, 2013.
(60) Provisional application No. 61/861,565, filed on Aug. 2, 2013.
(60) Provisional application No. 61/912,172, filed on Dec. 5, 2013.
(60) Provisional application No. 62/279,579, filed on Jan. 15, 2016.

(51) Int. Cl. A61B 18/12; A61B 18/02; A61B 18/14

(52) U.S. Cl. CPC A61B 18/1206; A61B 2018/00577; A61B 18/14; A61B 18/02

(57) ABSTRACT

A system for destruction the cellular membranes of unwanted or cancerous tissue without denaturing the intra-cellular contents of the cells comprising the tissue, comprising a treatment probe configured to apply radio-frequency energy to a target tissue followed an injection of immunologic adjuvant drugs into the treatment area and an electric pulse generator, and, optionally, a cryomachine operatively coupled to said treatment probe. The treatment optionally comprises a cryoablative pre-cycle to pre-stress the target tissue, thereby reducing the amount of radio-frequency energy needed to achieve tumor membrane destruction, but without damaging the lymphatic or vascular antigen or tumor drainage systems through which the subsequent antitumor effects are enhanced.

### CROSS-REFERENCE TO RELATED APPLICATIONS

This application is a continuation-in-part of U.S. patent application Ser. No. 14/451,333, filed Aug. 4, 2014, which in turn derives priority from U.S. Provisional Patent Application No. 61/867,048, filed Aug. 17, 2013 and from U.S. Provisional Patent Application No. 61/861,565, filed Aug. 2, 2013, all of which are incorporated herein by reference. This application is also a continuation-in-part of U.S. patent application Ser. No. 15/102,120, filed Jun. 6, 2016, which is a national stage entry of PCT/US2014/068774, filed Dec. 5, 2014, and which in turn derives priority from U.S. Provisional Patent Application No. 61/912,172, filed Dec. 5, 2013, all of which are incorporated herein by reference. This application is also a continuation-in-part of U.S. patent application Ser. No. 16/070,072, filed Jul. 13, 2018, which is a U.S. national stage entry of PCT/US2017/013486, filed Jan. 13, 2017, which in turn derives priority from U.S. Provisional Patent Application No. 62/276,579, filed Jan. 15, 2016, all of which are incorporated herein by reference.

### BACKGROUND OF THE INVENTION

### 1. Field of the Invention

The present invention relates generally to medical devices and treatment methods, and more particularly, to a device and method of utilizing radio frequency electrical membrane breakdown (“RFEMB”) and/or cryoablation followed by an RFEMB treatment protocol (“CRYO/EMB”) to treat unwanted soft and/or cancerous tissue, and to use the immediate tumor necrosis caused by RFEMB/cryoablation in connection with the intra-tumoral injection of a specially formulated mixture of immunostimulatory drugs to enhance the patient's immune response to the treated cells.

### 2. Background of the Invention

Cancer is not one single disease but rather a group of diseases with common characteristics that often result in sustained cell proliferation, reduced or delayed cell mortality, cooption of bodily angiogenesis and metabolic processes and evasion of bodily immune response which results in undesirable soft tissue growths called neoplasms or, more commonly, tumors. Removal or destruction of this aberrant tissue is a goal of many cancer treatment methods and modalities. Surgical tumor excision is one method of accomplishing this goal. Tissue ablation is another, minimally invasive method of destroying undesirable tissue in the body, and has been generally divided into thermal and non-thermal ablation technologies. Thermal ablation encompasses the addition and/or removal of heat to destroy undesirable cells. Cryoablation is a well established thermal ablation technique that kills cells by freezing of the tissue resulting in cell dehydration beginning at −15 C and by intracellular ice formation causing membrane rupture occurring at colder temperatures. Because cryoablative techniques can rupture cell membranes without denaturing cell proteins under certain conditions, such techniques have the additional ability to stimulate antitumor immune responses to thermolabile antigens in the patient.

Heat based techniques are also well established for ablation of both cancerous and non-cancerous tissues and include radio-frequency (RF) thermal, microwave and high intensity focused ultrasound ablation which raise localized tissue temperatures well above the body's normal 37° C. These methods use various techniques to apply energy to the target cells to raise interstitial temperature. For example, RF thermal ablation uses a high frequency electric field to induce vibrations in the cell membrane that are converted to heat by friction. Cell death occurs in as little as thirty (30) seconds once the cell temperature reaches 50° C. and increases as the temperature rises. At 60° C. cell death is instantaneous. If the intracellular temperature rises to between about 60 and 95° C., the mechanisms involved in cell death include cellular desiccation and protein coagulation. When the intracellular temperature exceeds 100° C., cellular vaporization occurs as intracellular water boils to steam. In the context of tissue ablation, cell temperatures not exceeding 50° C. are not considered clinically significant. Because cellular proteins are denatured by the heat of thermal ablation techniques, they may not be available to stimulate a specific immune response as they may be with cryoablation. Prior art heat-based techniques suffer from the drawback that they have little or no ability to spare normal structures in the treatment zone and so can be contraindicated based on tumor location or lead to complications from collateral injury.

Non-thermal ablation techniques include electrochemotherapy and irreversible electroporation (IRE), which although quite distinct from one another, each rely on the phenomenon of electroporation. With reference to FIG. 1, electroporation refers to the fact that the plasma membrane of a cell exposed to high voltage pulsed electric fields within certain parameters, becomes temporarily permeable due to destabilization of the lipid bilayer and the formation of pores P. The cell plasma membrane consists of a lipid bilayer with a thickness t of approximately 5 nm. With reference to FIG. 2(A), the membrane acts as a nonconducting, dielectric barrier forming, in essence, a capacitor. Physiological conditions produce a natural electric potential difference due to charge separation across the membrane between the inside and outside of the cell even in the absence of an applied electric field. This resting transmembrane voltage potential ranges from 40 mv for adipose cells to 85 mv for skeletal muscle cells and 90 mv for cardiac muscle cells and can vary by cell size and ion concentration, among other things.

With continued reference to FIGS. 2(B)-2(D), exposure of a cell to an externally applied electric field E induces an additional voltage V across the membrane as long as the external field is present. The induced transmembrane voltage is proportional to the strength of the external electric field and the radius of the cell. Formation of transmembrane pores P in the membrane occurs if the cumulative resting and applied transmembrane potential exceeds the threshold voltage which may typically be between 200 mV and 1 V. Poration of the membrane is reversible if the transmembrane potential does not exceed the critical value such that the pore area is small in relation to the total membrane surface. In such reversible electroporation, the cell membrane recovers after the applied field is removed and the cell remains viable. Above a critical transmembrane potential and with longer exposure times, poration becomes irreversible leading to eventual cell death due an influx of extracellular ions resulting in loss of homeostasis and subsequent apoptosis. Pathology after irreversible electroporation of a cell does not show structural or cellular changes until 24 hours after field exposure except in certain very limited tissue types. However, in all cases the mechanism of cellular destruction and death by IRE is apoptotic which requires considerable time to pass and is not visible pathologically in a time frame to be clinically useful in determining the efficacy of IRE treatment, which is an important clinical drawback to the method.

Developed in the early 1990's, electrochemotherapy combines the physical effect of reversible cell membrane poration with administration of chemotherapy drugs such as cisplatin and bleomycin. By temporarily increasing the cell membrane permeability, uptake of non-permeant or poorly permeant chemotherapeutic drugs is greatly enhanced. After the electric field is discontinued, the pores close and the drug molecules are retained inside the target cells without significant damage to the exposed cells. This approach to chemotherapy grew out of earlier research developing electroporation as a technique for transfection of genes and DNA molecules for therapeutic effect. In this context, irreversible electroporation leading to cell death was viewed as a failure in as much as the treated cells did not survive to realize the modification as intended.

IRE as an ablation method grew out of the realization that the “failure” to achieve reversible electroporation could be utilized to selectively kill undesired tissue. IRE effectively kills a predictable treatment area without the drawbacks of thermal ablation methods that destroy adjacent vascular and collagen structures. During a typical IRE treatment, one to three pairs of electrodes are placed in or around the tumor. Electrical pulses carefully chosen to induce an electrical field strength above the critical transmembrane potential are delivered in groups of ten, usually for nine cycles. Each ten-pulse cycle takes about one second, and the electrodes pause briefly before starting the next cycle. As described in U.S. Pat. No. 8,048,067 to Rubinsky, et al. and U.S. patent application Ser. No. 13/332,133 to Arena, et al. which are incorporated here by reference, the field strength and pulse characteristics are chosen to provide the necessary field strength for IRE but without inducing thermal effects as with RF thermal ablation.

However, the DC pulses used in currently available IRE methods and devices have characteristics that can limit their use or add risks for the patient because current methods and devices create severe muscle contraction during treatment. This is a significant disadvantage because it requires that a patient be placed and supported under general anesthesia with neuromuscular blockade in order for the procedure to be carried out, and this carries with it additional substantial inherent patient risks and costs. Moreover, since even relatively small muscular contractions can disrupt the proper placement of IRE electrodes, the efficacy of each additional pulse train used in a therapy regimen may be compromised without even being noticed during the treatment session. In addition, the high voltage DC pulses used by IRE may cause sparks to occur at the junction of the electrode and its insulation. These sparks can be of such an intensity as to cause a physical disruption of tissue leading to local complications.

Cancer cells produce antigens, which the immune system can use to identify and destroy them. These antigens are taken up by dendritic cells, which present the antigens to T lymphocytes in secondary lymphoid tissues (including lymph nodes). This can ultimately elicit either humoral (antibody) or cellular responses to the presented antigens by activating T cells to differentiate and proliferate into either helper T lymphocytes or cytotoxic T lymphocytes (CTLs). The T cells can then recognize the cancer cells by those antigens and destroy them directly or indirectly, through the participation of other components of the immune system. However, immediately following T cell activation by dendritic cells, the T cells begin to produce an inhibitory receptor, cytotoxic T lymphocyte-associated antigen 4 (CTLA-4), which, by binding to its ligand (B7) on the dendritic cells, dampens its activation state and limits its ability to contribute to antitumor immune responses. Similarly, activated T cells produce a second inhibitory receptor, Programmed Death-1 (PD-1), which downregulates their antitumor responses when bound to their cellular ligands PD-L1 or PD-L2, which are often expressed by cancer cells. Binding of cancer cell PD-1 ligand(s) to PD-1 on the activated T cells interrupts the immunological destruction of the cancer cells and allows the cancer cells to survive. See Antoni Ribas, “Tumor immunotherapy directed at PD-1”, New England Journal of Medicine 366 (26): 2517-9 (28 Jun. 2012).

Approaches to modulate this tumor immune response, in general, are now available and have been shown to have positive effects, improving patient survival, in certain tumor types. One of these approaches utilizes Sipuleucel-T (Provenge), which uses autologous patient dendritic cells activated with a GM-CSF-PAP fusion protein infused back into the patient, has been shown in multiple studies to improve survival in hormone resistant prostate cancer by an average of approximately 4 months. Sipuleucel-T showed overall survival (OS) benefit to patients in three double-blind randomized phase III clinical trials.

Ipilimumab, marketed as Yervoy, is a human monoclonal antibody and works by blocking the CTLA-4 inhibitory signal, resulting in an elevated state of activation, allowing the CTLs to destroy the cancer cells. CTLA-4 (also known as CD152) is expressed on the surface of T cells along with the co-stimulatory receptor CD28. In contrast to CD28, which activates T cells when bound to B7 on antigen presenting cells (APCs), CTLA-4 interferes with IL-2 production, IL-2 receptor expression, interrupts cell cycle progression of activated T cells, and antagonizes T cell activation. Inhibition of CTLA-4 receptors using ipilimumab reportedly resulted in increased activity of T cells and led to tumor regression. Studies have shown ipilimumab to improve survival in patients with metastatic melanoma, but ipilimumab alone has been shown to be unsuccessful as a single agent in, e.g., pancreatic cancer. See Royal R E, Levy C, Turner K et al., “Phase 2 trial of single agent ipilimumab (anti-CTLA-4) for locally advanced or metastatic pancreatic adenocarcinoma”, J Immunother. 2010 October; 33(8):828-33.

A third class of drug with immunomodulatory effects is Tasquinimod. Tasquinimod is a novel small molecule that targets the tumor microenvironment by binding to S100A9 and modulating regulatory myeloid cell functions, exerting immunomodulatory, anti-angiogenic and anti-metastatic properties. Tasquinimod may also suppress the tumor hypoxic response, contributing to its effect on the tumor microenvironment. It has been shown to have significant clinical effects in, e.g., castrate resistant prostate cancer.

Because cells ablated by IRE methods undergo apoptotic death without membrane rupture and concomitant release of intracellular antigens, their ability to induce a supplemental immune response as observed with cryoablation is impaired. When used as the sole ablative tool in a treatment protocol, IRE's inability to induce a supplemental immune response is a substantial limitation to its therapeutic benefit for patients.

On the other hand, prior art cryoablation techniques are limited by clinical disadvantages arising from the extreme cold and its capacity to destroy nearby critical healthy structures, such as collagen networks and microvasculature.

Thus, a treatment method that does not need neuromuscular blockade, spares tissue structure, does not cause potentially dangerous sparking and produces an immunologic response would provide an excellent means for treating unwanted tissue.

For the treatment of all of the above conditions, what is needed is a minimally invasive tissue treatment technology that can avoid damaging healthy tissue while exposing cellular contents without denaturing such cellular contents so that they can trigger a clinically useful immune response.

In addition, a treatment method that can be accurately targeted at previously identified unwanted tissue, and that spares tissue structure outside of the focal treatment area, would be advantageous.

It would also be advantageous to provide a system and method for carrying out this treatment on an outpatient setting under local anesthesia, using a method that does not require general anesthesia or a neuromuscular blockade, where appropriate, or alternatively on an inpatient intraoperative basis optionally utilizing an open, laparoscopic or robotic access to the treatment area.

### SUMMARY OF THE INVENTION

It is, therefore, an object of the present invention to provide a method for the treatment of unwanted soft and/or cancerous tissue using electrical pulses which causes immediate cell death through the mechanism of complete breakdown of the membranes of the target cells, accompanied by the intra-tumoral injection of a specially formulated mixture of immunostimulatory drugs to enhance the patient's immune response to antigens released by the treated cells.

It is another object of the present invention to provide a method for the treatment of unwanted soft and/or cancerous tissue using a CRYO/EMB technique whereby an initial cryoablation cycle pre-stresses the target cell membrane, thereby reducing the amount of radio-frequency energy needed to achieve tumor membrane destruction, but without damaging the lymphatic or vascular antigen or tumor drainage systems through which the subsequent antitumor effects are enhanced.

It is yet another object of the present invention to provide such a treatment method that does not require the administration of general anesthesia or a neuromuscular blockade to the patient.

The present invention includes an imaging, guidance, planning and treatment system integrated into a single unit or assembly of components, and a method for using same, that can be safely and effectively deployed to treat all forms of unwanted soft and/or cancerous tissue. The system utilizes the novel process of Radio-Frequency Electrical Membrane Breakdown (“EMB” or “RFEMB”) to destroy the cellular membranes of unwanted or cancerous tissue without denaturing the intra-cellular contents of the cells comprising the tissue, thereby exposing tumor antigens and other intra-cellular components which can have an immunologic effect on local or distant cancerous tissue, with or without the addition of immunologic adjuvant drugs.

In preferred embodiments, the system also utilizes an optimized cryoablation or cooling pre-cycle to “pre-stress” the cell membrane to increase the efficacy of, and reduce the length of treatment under, a subsequent RFEMB protocol. The use of a cryoablation pre-cycle before the application of a RFEMB treatment protocol cools and “hardens” the membrane of the target cells such that the EMB treatment protocol, which, as described below, achieves complete cellular membrane breakdown via the application of electrical energy optimized to rapidly flex the target cell membrane such that it forcibly “snaps”, or ruptures, requires a shorter period of energy application in the form of fewer electrical pulses applied to the target tissue. This in turn eliminates one or more pauses to the EMB treatment protocol to dissipate any heat accumulated at the target site during treatment, thereby reducing the overall length of the treatment without sacrificing efficacy.

The use of EMB to achieve focal tumor treatment with an enhanced immunologic effect on surrounding cancerous tissue is disclosed in U.S. patent application Ser. Nos. 14/451,333 and 15/102,120, International Patent Application Nos. PCT/US14/68774, PCT/US16/16300, PCT/US16/16352, PCT/US16/16955, PCT/US16/16501 and PCT/US16/15944, which are all fully incorporated herein by reference. In addition, the use of cryoablative techniques in combination with EMB to achieve focal tumor treatment with an enhanced immunologic effect is also disclosed in International Patent Application No., PCT/US17/13486, which is also incorporated herein by reference.

EMB is the application of an external oscillating electric field to cause vibration and flexing of the cell membrane, which results in a dramatic and immediate mechanical tearing, disintegration and/or rupturing of the cell membrane. Unlike the IRE process, in which nano-pores are created in the cell membrane but through which little or no content of the cell is released, the EMB protocol completely tears open the cell membrane such that the entire contents of the cell are expelled into the extracellular fluid, and internal components of the cell membrane itself are exposed. EMB achieves this effect by applying specifically configured electric field profiles, comprising significantly higher energy levels (as much as 100 times greater) as compared to the IRE process, to directly and completely disintegrate the cell membrane rather than to electroporate the cell membrane. Such electric field profiles are not possible using currently available IRE equipment and protocols. The inability of current IRE methods and energy protocols to deliver the energy necessary to cause EMB explains why IRE treated specimens have never shown the pathologic characteristics of EMB-treated specimens, and is a critical reason why EMB had not until now been recognized as an alternative method of cell destruction.

The system according to the present invention comprises a software and hardware system, and method for using the same, for detecting and measuring a mass of target tissue in a treatment area of a patient, for designing an EMB and/or CRYO/EMB treatment protocol to treat said mass, either of which are preferably utilized in connection with an intra-tumoral constant volume infusion of an immunologic response-enhancing drug mixture, and for applying said treatment protocol in an outpatient, doctor's office, or intra-operative hospital setting. The system includes an EMB pulse generator 16, one or more EMB treatment probes 20, one or more cryoablation needles, and one or more injection needles (in preferred embodiments, two or more of the foregoing instruments are combined into a single cryo/EMB treatment probe) for targeted delivery of cryo/EMB or EMB treatment and optionally an immunologic response enhancing biologic drug mixture. The inventive system may also utilize additional probes or devices such as one or more trackable biopsy needles 200 and one or more temperature probes 22, which in preferred embodiments may be inserted through a central lumen of the aforementioned EMB and/or cryo/EMB treatment probe, as will be described. In certain embodiments, the system further employs a software-hardware controller unit (SHCU) operatively connected to said generator 16, probes 20, cryoablation needles, injection needles, and optional biopsy needles 200 and temperature probe(s) 22, along with one or more additional optional devices such as trackable anesthesia needles 300, endoscopic imaging scanners, ultrasound scanners, and/or other imaging devices or energy sources, and operating software for controlling the operation of each of these hardware devices.

### BRIEF DESCRIPTION OF THE DRAWINGS

FIG. 1 is a diagram of a cell membrane pore.

FIG. 2 is a diagram of cell membrane pore formation by a prior art method.

FIG. 3 is a schematic diagram of the software and hardware system according to the present invention.

FIG. 4A is a comparison of a prior art charge reversal with an instant charge reversal according to the present invention.

FIG. 4B is a square wave from instant charge reversal pulse according to the present invention.

FIG. 5 is a diagram of the forces imposed on a cell membrane as a function of electric field pulse width according to the present invention.

FIG. 6 is a diagram of a prior art failure to deliver prescribed pulses due to excess current.

FIG. 7A is a schematic diagram depicting a TRUSS scan of a suspect tissue mass.

FIG. 7B is a schematic diagram depicting the results of a 3D Fused Image of a suspect tissue mass.

FIG. 8 is a schematic diagram depicting the target treatment area and Predicted Ablation Zone relative to a therapeutic EMB treatment probe 20 prior to delivering treatment.

FIG. 9 is a schematic diagram of a pulse generation and delivery system for application of the method of the present invention.

FIG. 10 is a diagram of the parameters of a partial pulse train according to the present invention.

FIG. 11 is a schematic diagram depicting the target treatment area and Predicted Ablation Zone relative to a therapeutic EMB treatment probe 20 at the start of treatment delivery.

FIG. 12A is a schematic diagram of a therapeutic EMB treatment probe 20 according to one embodiment of the present invention.

FIG. 12B is a composite schematic diagram (1, 2 and 3) of the therapeutic EMB treatment probe 20 of FIG. 12A showing insulating sheath 23 in various stages of retraction.

FIG. 12C is a composite schematic diagram (1 and 2) of a therapeutic EMB treatment probe 20 according to another embodiment of the present invention.

FIG. 12D is a composite schematic diagram (1 and 2) of the therapeutic EMB treatment probe 20 of FIG. 12C showing insulating sheath 23 in various stages of retraction.

FIG. 13 is a schematic diagram of the enhanced trackable biopsy needle 200 according to the present invention.

FIG. 14 is a schematic diagram of the enhanced trackable anesthesia needle 300 according to the present invention.

FIG. 15 is a schematic diagram depicting the positioning of a therapeutic EMB treatment probe 20 according to an embodiment of the present invention proximate the treatment area 2.

FIG. 16 is a schematic diagram depicting the positioning of a therapeutic EMB treatment probe 20 comprising a thermocouple 7 according to another embodiment of the present invention proximate the treatment area 2.

FIG. 17 is a schematic diagram depicting the positioning of a therapeutic EMB treatment probe 20 comprising a side port 8 for exposure of needle 9 according to another embodiment of the present invention proximate the treatment area 2.

FIG. 18 is a schematic diagram depicting the positioning of a therapeutic EMB treatment probe 20 comprising a unipolar electrode 11 according to another embodiment of the present invention proximate the treatment area 2.

FIG. 19 is a schematic diagram depicting the positioning of a therapeutic EMB treatment probe 20 comprising a side port 8 for exposure of electrode-bearing needle 17 according to another embodiment of the present invention proximate the treatment area 2.

FIG. 20 is a schematic diagram depicting the positioning of a therapeutic EMB treatment probe 20 according to another embodiment of the present invention inside a cavity 400 in the human body.

FIG. 21 is a schematic diagram depicting the positioning of a therapeutic EMB treatment probe 20 comprising an expandable stabilizing balloon 27 according to another embodiment of the present invention inside a cavity 400 in the human body.

FIG. 22 is a schematic diagram depicting the positioning of a therapeutic EMB treatment probe 20 comprising an expandable electrode-bearing balloon 27 according to another embodiment of the present invention inside a cavity 400 in the human body.

FIG. 23 is a schematic diagram depicting the positioning of a therapeutic EMB treatment probe 20 according to another embodiment of the present invention inside a cavity 400 in the human body.

FIG. 24 is a schematic diagram depicting the use of two therapeutic EMB treatment probes 20 for delivery of EMB treatment.

FIG. 25 is a schematic diagram depicting the positioning of a therapeutic EMB treatment probe 20 delivered endoscopically using endoscopic ultrasound as a guidance method according to another embodiment of the present invention.

FIG. 26 is a schematic diagram depicting the positioning of a therapeutic EMB treatment probe 20 delivered endoscopically using endoscopic ultrasound as a guidance method according to another embodiment of the present invention.

FIG. 27 is a schematic diagram depicting the positioning of a therapeutic EMB treatment catheter type probe 20 comprising an ultrasound transducer according to another embodiment of the present invention proximate the treatment area 2.

FIG. 28 is a schematic diagram depicting the positioning of a therapeutic EMB treatment catheter type probe 20 wherein needle 9 exits the distal end of catheter probe 20 according to another embodiment of the present invention proximate the treatment area 2.

FIG. 29 is a composite (A & B) schematic diagram depicting the positioning of a therapeutic EMB treatment probe 20 comprising an inflatable stent 19 according to another embodiment of the present invention inside a cavity 400 in the human body.

FIG. 30 is a schematic diagram depicting the positioning of a stent 19 left by EMB treatment probe 20 inside a cavity 400 in the human body.

FIG. 31 is a schematic diagram depicting a US scan of a suspect tissue mass.

FIG. 32 is a schematic diagram depicting the results of a 3D Fused Image of a suspect tissue mass.

FIG. 33 is a schematic diagram depicting the target treatment area and Predicted Ablation Zone relative to a therapeutic EMB treatment probe 20 prior to delivering treatment.

FIG. 34 is a schematic diagram depicting the target treatment area and Predicted Ablation Zone relative to a therapeutic EMB treatment probe 20 at the start of treatment delivery.

FIG. 35 is a schematic diagram depicting a US scan of a suspect tissue mass with the EMB catheter probe 20 with integrated US being moved into place.

FIG. 36 is a schematic diagram depicting the results of a 3D Fused Image of a suspect tissue mass.

FIG. 37 is a schematic diagram depicting the target treatment area and Predicted Ablation Zone relative to a therapeutic EMB treatment probe 20 prior to delivering treatment.

FIG. 38 is a schematic diagram depicting the target treatment area and Predicted Ablation Zone relative to a therapeutic EMB treatment probe 20 at the start of treatment delivery.

FIG. 39 is a schematic diagram depicting the positioning of a therapeutic EMB treatment catheter type probe 20 comprising an ultrasound transducer according to another embodiment of the present invention proximate the treatment area 2.

FIG. 40 is a schematic diagram depicting the positioning of a therapeutic EMB treatment catheter type probe 20 wherein needle 9 exits the distal end of catheter probe 20 according to another embodiment of the present invention proximate the treatment area 2.

FIG. 41 is a schematic diagram depicting a device having two cryoprobe electrodes and the ability to deliver electrical pulses and create reversible electroporation.

FIG. 42 is a schematic diagram depicting a device having one cryoprobe electrode and one non-cryoprobe electrode.

FIG. 43 is a schematic diagram depicting an embodiment of a device having one cryoprobe and two retractable electrode needles.

FIG. 44 is a schematic diagram depicting an embodiment of a device having one cryoprobe and two retractable electrode needles configured to inject plasmids, a biologic drug formulation and/or immunostimulatory drug mixture.

FIG. 45 is a schematic diagram depicting an embodiment of a device having one cryoprobe and retractable electrode needles configured to inject plasmids, a biologic drug formulation and/or immunostimulatory drug mixture.

FIG. 46 is a schematic diagram depicting an embodiment of a device having one cryoprobe and two electrodes on the single cryoprobe.

FIG. 47 is a schematic diagram depicting an embodiment of a device having one cryoprobe electrode and one indifferent electrode.

FIG. 48 is a schematic diagram depicting an embodiment of a device having a cryoprobe treatment portion detachable from an electric therapy delivery portion.

FIG. 49 shows an embodiment according to the present invention whereby a multi-tine needle is used to inject one or more immunologic enhancing drugs.

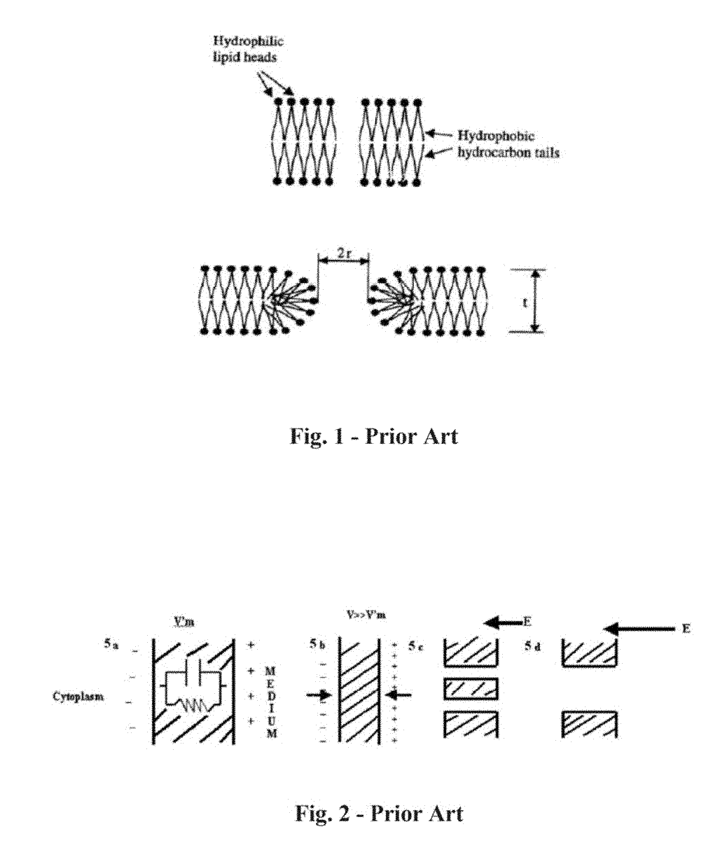

### DETAILED DESCRIPTION

In general, the software-hardware controller unit (SHCU) operating the proprietary treatment system software according to the present invention facilitates the treatment of cancerous or other unwanted tissue by directing the placement of cryoablation needles, EMB treatment probe(s) 20, and injection needles (or a single probe to capture one or all of the foregoing functionalities) and by delivering electric pulses designed to cause EMB within the target tissue to EMB treatment probe(s) 20, by delivering and/or directing the delivery of a cryoablation treatment protocol via one or more cryoablation needles, and by delivering and/or directing the delivery of a combination biologic drug formulation as described herein via one or more injection needles. A combination biologic drug formulation and/or immunostimulatory drug mixture as described herein may be applied as a depot or other form of injectable as the application requires. In a preferred embodiment, at least the herein-described EMB or cryo/EMB treatment is delivered via a single probe that is placed robotically or manually, wherein the SHCU collects measurements as needed and/or receives input to determine the optimal cryo/EMB or EMB treatment protocol and then delivers said protocol via the single probe automatically, including cryoablation, EMB treatment and/or cooling cycles as necessary, without the need for further intervention.

In certain embodiments, the SHCU also directs the placement of optional components such as biopsy needle(s) 200 and anesthesia needle(s) 300.

In further embodiments, the SHCU enables monitoring of the treatment protocol in real time via one or more two- or three-dimensional imaging devices and via one or more biopsy samples taken at strategic locations to measure treatment efficacy. The system is such that the treatment may be performed by a physician under the guidance of the software, or may be performed completely automatically, from the process of imaging the treatment area to the process of placing one or more probes using robotic arms operatively connected to the SHCU to the process of delivering cryoablative therapy, electric pulses and/or high pressure infusions and monitoring the results of same. Specific components of the invention will now be described in greater detail.

Further, the method described herein may be performed without the aid of the herein-described SHCU or any other control software or device, using the novel cryo/EMB treatment probe described herein.

EMB Pulse Generator 16

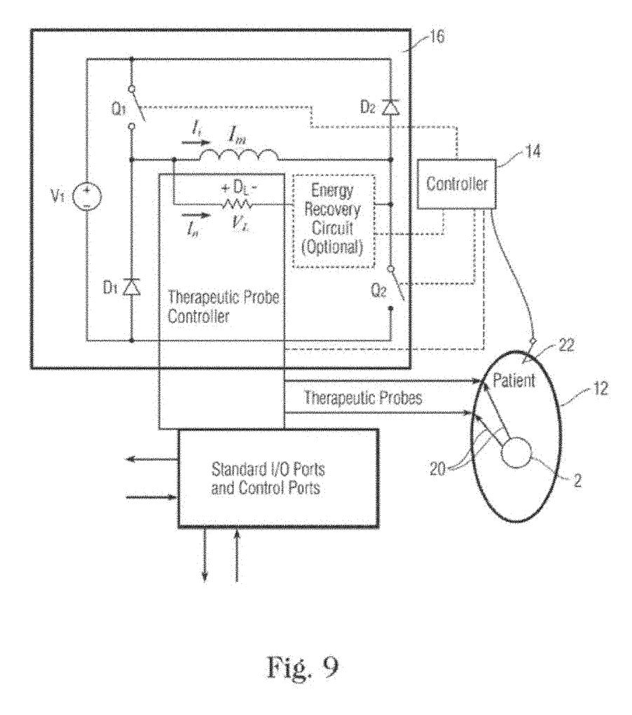

FIG. 9 is a schematic diagram of a system for generation of the electric field necessary to induce EMB of cells 2 within a patient 12. The system includes the EMB pulse generator 16 which, in certain embodiments, is operatively coupled to Software Hardware Control Unit (SHCU) 14 for controlling generation and delivery to the EMB treatment probes 20 (two are shown) of the electrical pulses necessary to generate an appropriate electric field to achieve EMB. FIG. 9 also depicts optional onboard controller 15 which is preferably the point of interface between EMB pulse generator 16 and SHCU 14. Thus, onboard controller 15 may perform functions such as accepting triggering data from SHCU 14 for relay to pulse generator 16 and providing feedback to the SHCU regarding the functioning of the pulse generator 16. The various treatment probes (described in greater detail below) are placed in proximity to the soft tissue or cancerous cells 2 which are intended to be treated through the process of EMB and/or CRYO/EMB and the bipolar pulses are shaped, designed and applied to achieve that result in an optimal fashion. A temperature probe 22 may be provided for percutaneous temperature measurement and feedback to the controller of the temperature at, on or near the electrodes. The controller may preferably include an onboard digital processor and a memory and may be a general purpose computer system, programmable logic controller or similar digital logic control device. The controller is preferably configured to control the signal output characteristics of the signal generation including the voltage, frequency, shape, polarity and duration of pulses comprising the EMB treatment protocol as well as the total number of pulses delivered in a pulse train and the duration of the inter pulse burst interval. The controller is also preferably configured to control the output of a cryogenic freezing unit as will be described herein.

With continued reference to FIG. 9, the EMB protocol calls for a series of short and intense bi-polar electric pulses delivered from the pulse generator through one or more EMB treatment probes 20 inserted directly into, or placed around the target tissue 2. The bi-polar pulses generate an oscillating electric field between the electrodes that induce a similarly rapid and oscillating buildup of transmembrane potential across the cell membrane. The built up charge applies an oscillating and flexing force to the cellular membrane which upon reaching a critical value causes rupture of the membrane and spillage of the cellular content. Bipolar pulses are more lethal than monopolar pulses because the pulsed electric field causes movement of charged molecules in the cell membrane and reversal in the orientation or polarity of the electric field causes a corresponding change in the direction of movement of the charged molecules and of the forces acting on the cell. The added stresses that are placed on the cell membrane by alternating changes in the movement of charged molecules create additional internal and external changes that cause indentations, crevasses, rifts and irregular sudden tears in the cell membrane causing more extensive, diverse and random damage, and disintegration of the cell membrane.

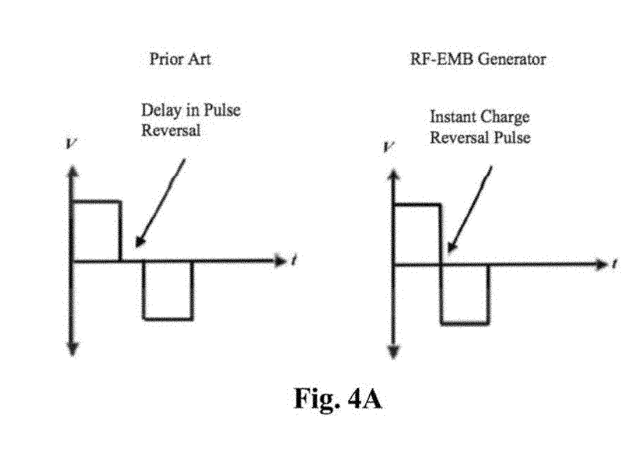

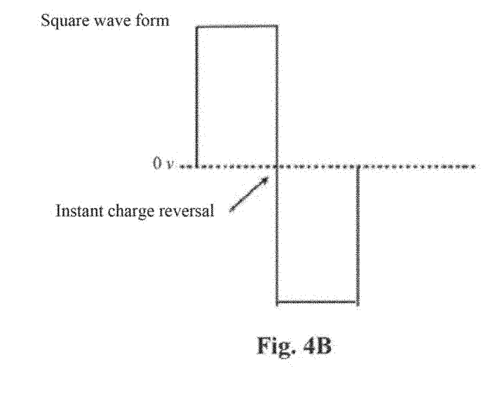

With reference to FIG. 4B, in addition to being bi-polar, the preferred embodiment of electric pulses is one for which the voltage over time traces a square wave form and is characterized by instant charge reversal pulses (ICR). A square voltage wave form is one that maintains a substantially constant voltage of not less than 80% of peak voltage for the duration of the single polarity portion of the trace, except during the polarity transition. An instant charge reversal pulse is a pulse that is specifically designed to ensure that substantially no relaxation time is permitted between the positive and negative polarities of the bi-polar pulse (See FIG. 4A). That is, the polarity transition happens virtually instantaneously.

The destruction of dielectric cell membranes through the process of Electrical Membrane Breakdown is significantly more effective if the applied voltage pulse can transition from a positive to a negative polarity without delay in between. Instant charge reversal prevents rearrangement of induced surface charges resulting in a short state of tension and transient mechanical forces in the cells, the effects of which are amplified by large and abrupt force reversals. Alternating stress on the target cell that causes structural fatigue is thought to reduce the critical electric field strength required for EMB. The added structural fatigue inside and along the cell membrane results in or contributes to physical changes in the structure of the cell. These physical changes and defects appear in response to the force applied with the oscillating EMB protocol and approach dielectric membrane breakdown as the membrane position shifts in response to the oscillation, up to the point of total membrane rupture and catastrophic discharge. This can be analogized to fatigue or weakening of a material caused by progressive and localized structural damage that occurs when a material is subjected to cyclic loading, such as for example a metal paper clip that is subjected to repeated bending. The nominal maximum stress values that cause such damage may be much less than the strength of the material under ordinary conditions. The effectiveness of this waveform compared to other pulse waveforms can save up to ⅕ or ⅙ of the total energy requirement.

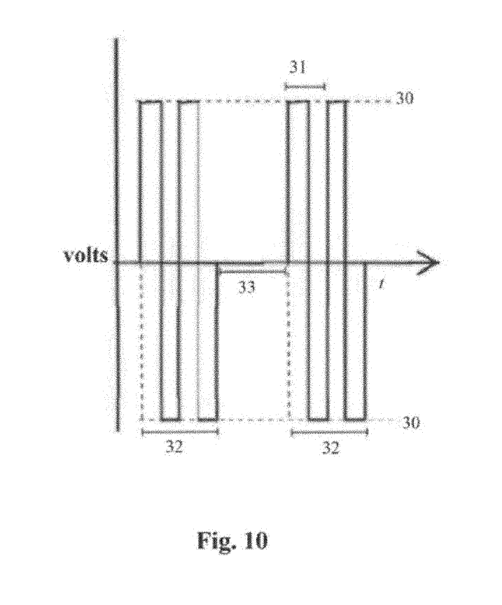

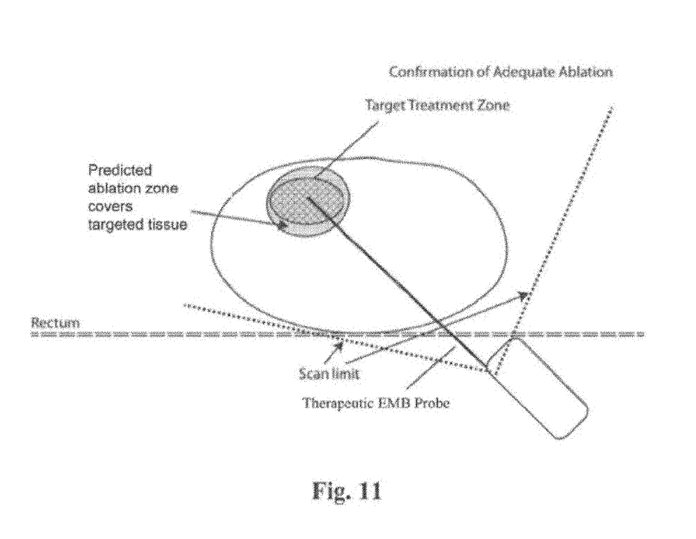

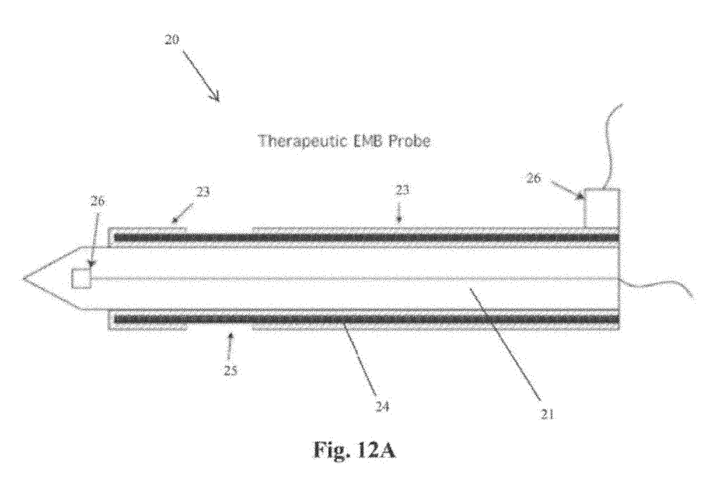

With reference to FIG. 10, another important characteristic of the applied electric field is the field strength (Volts/cm) which is a function of both the voltage 30 applied to the electrodes by the pulse generator 16 and the electrode spacing. Typical electrode spacing for a bi-polar, needle type probe might be 1 cm, while spacing between multiple needle probe electrodes can be selected by the surgeon and might typically be from 0.75 cm to 1.5 cm. A pulse generator for application of the present invention is capable of delivering up to a 10 kV potential. The actual applied field strength will vary over the course of a treatment to control circuit amperage which is the controlling factor in heat generation, and patient safety (preventing large unanticipated current flows as the tissue impedance falls during a treatment). Where voltage and thus field strength is limited by heating concerns, the duration of the treatment cycle may be extended to compensate for the diminished charge accumulation. Absent thermal considerations, a preferred field strength for EMB is in the range of 1,500 V/cm to 10,000 V/cm.

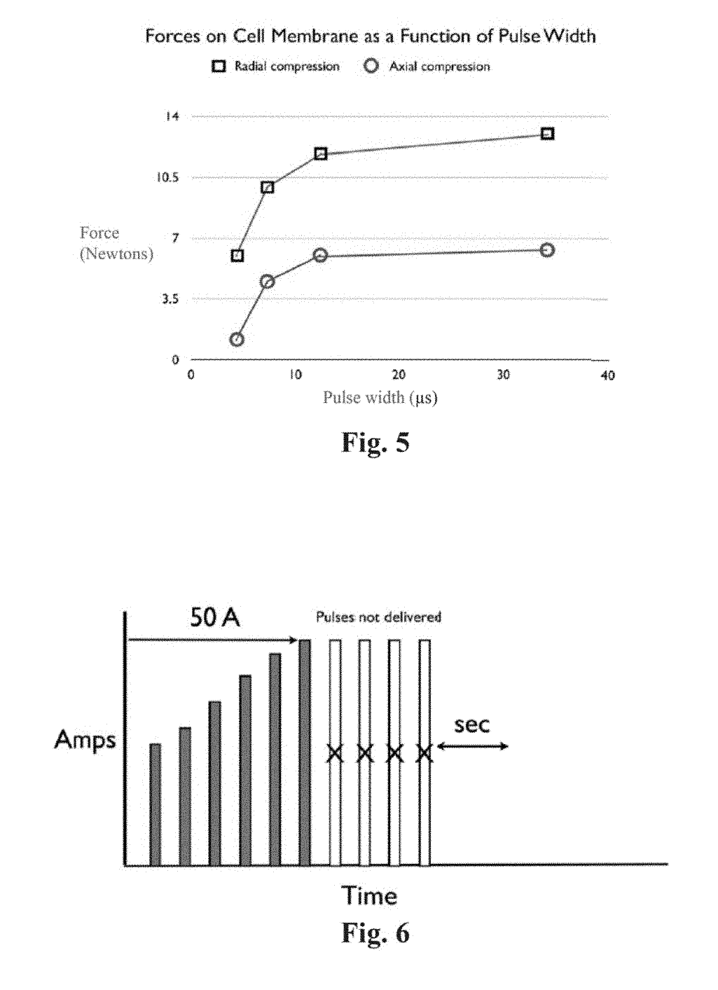

With continued reference to FIG. 10, the frequency 31 of the electric signal supplied to the EMB treatment probes 20, and thus of the field polarity oscillations of the resulting electric field, influences the total energy imparted on the subject tissue and thus the efficacy of the treatment but are less critical than other characteristics. A preferred signal frequency is from 14.2 kHz to less than 500 kHz. The lower frequency bound imparts the maximum energy per cycle below which no further incremental energy deposition is achieved. With reference to FIG. 5, the upper frequency limit is set based on the observation that above 500 kHz, the polarity oscillations are too short to develop enough motive force on the cell membrane to induce the desired cell membrane distortion and movement. More specifically, at 500 kHz the duration of a single full cycle is 2 μs of which half is of positive polarity and half negative. When the duration of a single polarity approaches 1 μs there is insufficient time for charge to accumulate and motive force to develop on the membrane. Consequently, membrane movement is reduced or eliminated and EMB does not occur. In a more preferred embodiment the signal frequency is from 100 kHz to 450 kHz. Here the lower bound is determined by a desire to avoid the need for anesthesia or neuromuscular-blocking drugs to limit or avoid the muscle contraction stimulating effects of electrical signals applied to the body. The upper bound in this more preferred embodiment is suggested by the frequency of radiofrequency thermal ablation equipment already approved by the FDA, which has been deemed safe for therapeutic use in medical patients.

In addition, the energy profiles that are used to create EMB also avoid potentially serious patient risks from interference with cardiac sinus rhythm, as well as localized barotrauma, which can occur with other therapies.

Cryoablation Unit

In preferred embodiments, the system also utilizes an optimized cryoablation or cooling pre-cycle to “pre-stress” the cell membrane to increase the efficacy of, and reduce the length of treatment under, a subsequent RFEMB protocol. The use of a cryoablation pre-cycle before the application of a RFEMB treatment protocol cools and “hardens” the membrane of the target cells such that the EMB treatment protocol requires a shorter period of energy application in the form of fewer electrical pulses applied to the target tissue. This in turn reduces the overall length of the treatment without sacrificing efficacy.

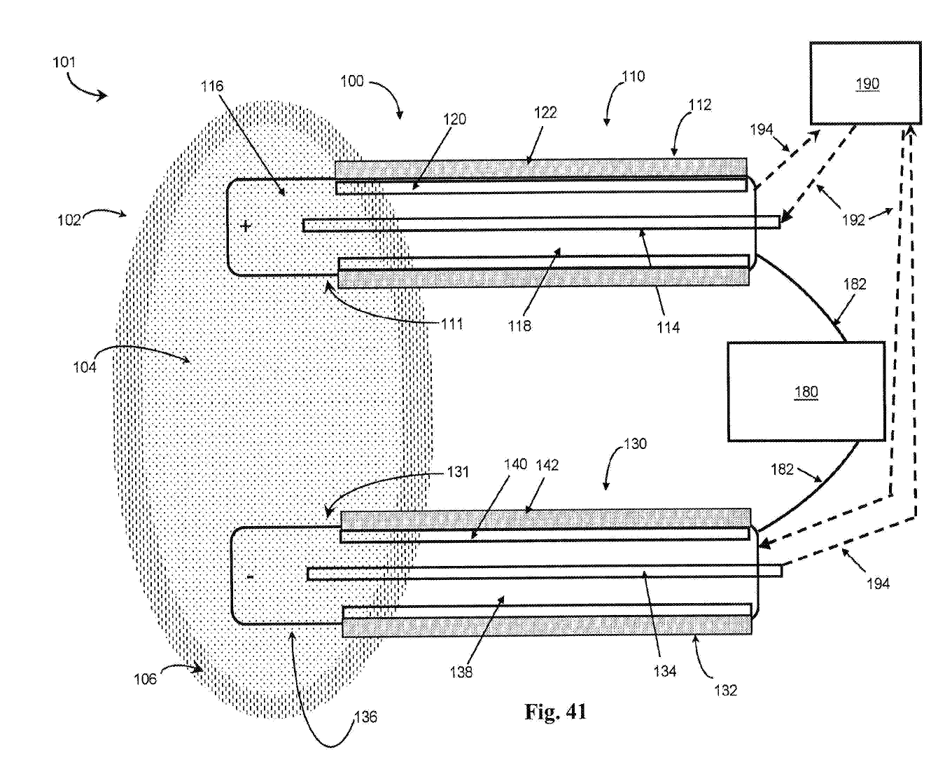

A cryofreezing unit or cryomachine 90 (see FIGS. 41-48) may be a device of the type known in the art and used for such purpose, including devices configured for use in prior art cryoablative techniques. Such a device must be capable of delivering cooled gas or liquid to the cryo or cryo/EMB treatment probe as described herein, and is operatively connected thereto and preferably also to the SHCU for automated cryo or cryo/EMB treatment application, also as described herein. The cryomachine 90 is described in further detail with reference to the cryo/EMB probes shown in FIGS. 41-48, below.

Treatment Probes 20

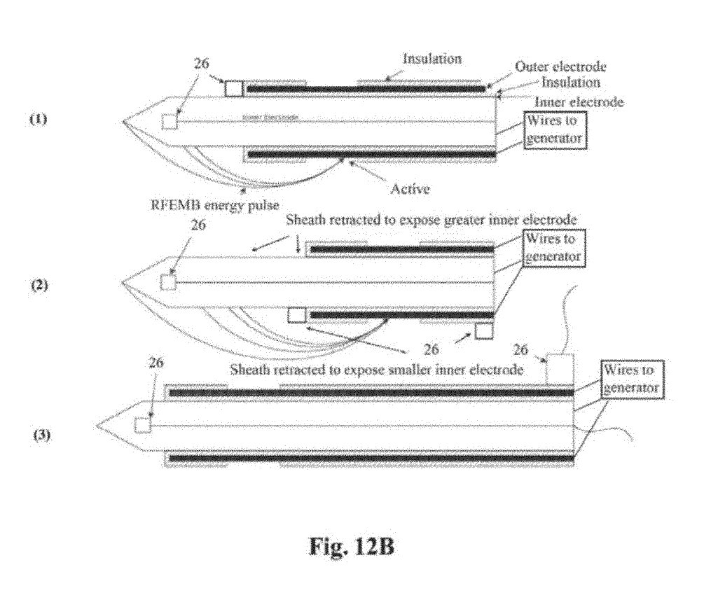

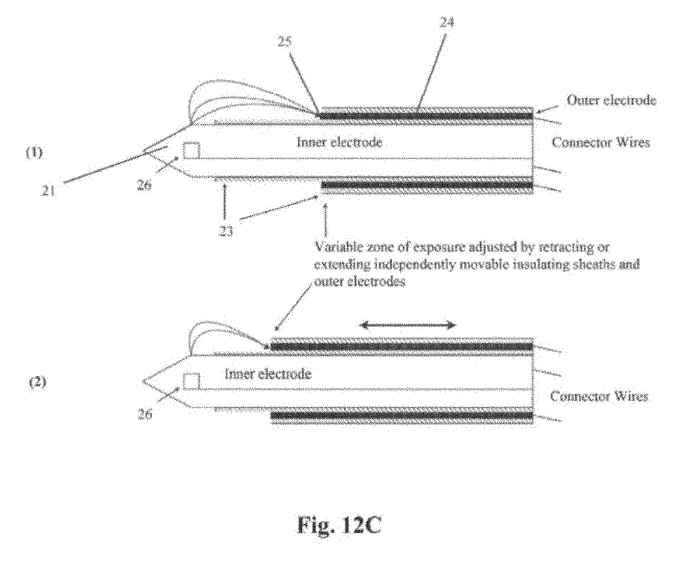

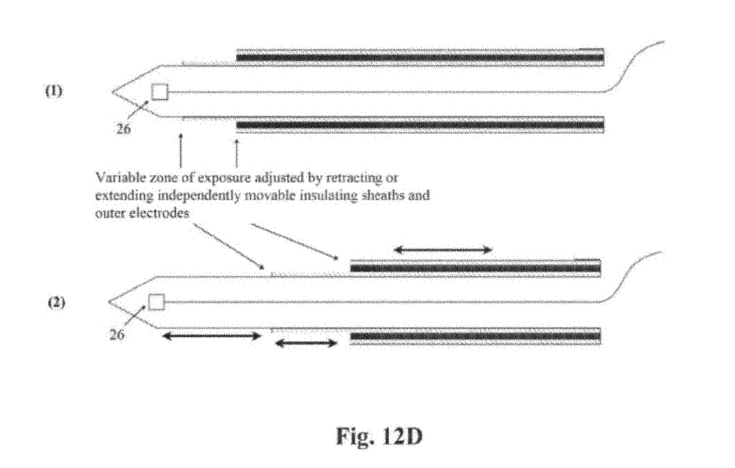

FIGS. 12A-12D depict a first set of embodiments of a therapeutic EMB treatment probe 20. With reference to FIGS. 12A-12B, the core (or inner electrode) 21 of EMB treatment probe 20 is preferably a needle of gage 17-22 with a length of 5-25 cm, and may be solid or hollow. Core 21 is preferably made of an electrically conductive material, such as stainless steel, and may additionally comprise one or more coatings of another conductive material, such as copper or gold, on the surface thereof. As shown in FIGS. 12A-12D, in the instant embodiment, the core 21 of treatment probe 20 has a pointed tip, wherein the pointed shape may be a 3-sided trocar point or a beveled point; however, in other embodiments, the tip may be rounded or flat. Treatment probe 20 further comprises an outer electrode 24 covering core 21 on at least one side. In a preferred embodiment, outer electrode 24 is also a cylindrical member completely surrounding the diameter of core 21. An insulating sheath 23, made of an inert material compatible with bodily tissue, such as Teflon® or Mylar®, is disposed around the exterior of core 21 and isolates core 21 from outer electrode 24. In this embodiment, insulating sheath 23 is also a cylindrical body surrounding the entire diameter of core 21 and completely encapsulating outer electrode 24 except at active area 25, where outer electrode 24 is exposed directly to the treatment area. In an alternate embodiment, shown in FIGS. 12C-12D, insulating sheath 23 comprises two solid cylindrical sheaths wherein the outer sheath completely encapsulates the lateral area of outer electrode 24 and only the distal end of outer electrode 24 is exposed to the treatment area as active area 25. Insulating sheath 23 and outer electrode 24 are preferably movable as a unit along a lateral dimension of core 21 so that the surface area of core 21 that is exposed to the treatment area is adjustable, thus changing the size of the lesion created by the EMB pulses. FIGS. 12B(3) and 12C(2) depict insulating sheath 23 and outer electrode 24 advanced towards the pointed tip of core 21, defining a relatively small treatment area, while FIGS. 12B(2) and 12C(1) depict insulating sheath 23 and outer electrode 24 retracted to define a relatively large treatment area. Electromagnetic (EM) sensors 26 on both core 21 and insulating sheath 23/outer electrode 24 member may send information to the Software Hardware Controller Unit (SHCU) for determining the relative positions of these two elements and thus the size of the treatment area, preferably in real time. EM sensors 26 may be a passive EM tracking sensor/field generator, such as the EM tracking sensor manufactured by Traxtal Inc. Alternatively, instead of utilizing EM sensors, EMB treatment probes 20 may be tracked in real time and guided using endoscopy, ultrasound or other imaging means known in the art.

One means for enabling the relative movement between core 21 and insulating sheath 23/outer electrode 24 member is to attach insulating sheath 23/outer electrode 24 member to a fixed member (i.e., a handle) at a distal end of probe 20 opposite the tip of core 21 by a screw mechanism, the turning of which would advance and retract the insulating sheath 23/outer electrode 24 member along the body of the core 21. Other means for achieving this functionality of EMB treatment probe 20 are known in the art.

One of conductive elements 21, 24 comprises a positive electrode, while the other comprises a negative electrode. Both core 21 and outer electrode 24 are connected to the EMB pulse generator through insulated conductive wires, and which are capable of delivering therapeutic EMB pulsed radio frequency energy or biphasic pulsed electrical energy under sufficient conditions and with sufficient treatment parameters to achieve the destruction and disintegration of the membranes of cancer cells, or unwanted tissue, through the process of EMB, as described in more detail above. The insulated connection wires may either be contained within the interior of EMB treatment probes 20 or on the surface thereof. However, EMB treatment probes 20 may also be designed to deliver thermal radio frequency energy treatment, if desired, as a complement to or instead of EMB treatment as described further herein.

Alternatively, or in addition to the sensors described above, EMB treatment probes 20 may contain a thermocouple, such as a Type K-40AWG thermocouple with Polyimide Primary/Nylon Bond Coat insulation and a temperature range of −40 to +180 C, manufactured by Measurement Specialties. The lumen of the optional thermocouple may be located on EMB treatment probe 20 such that the temperature at the tip of the probe can be monitored and the energy delivery to probe 20 modified to maintain a desired temperature at the tip of probe 20.

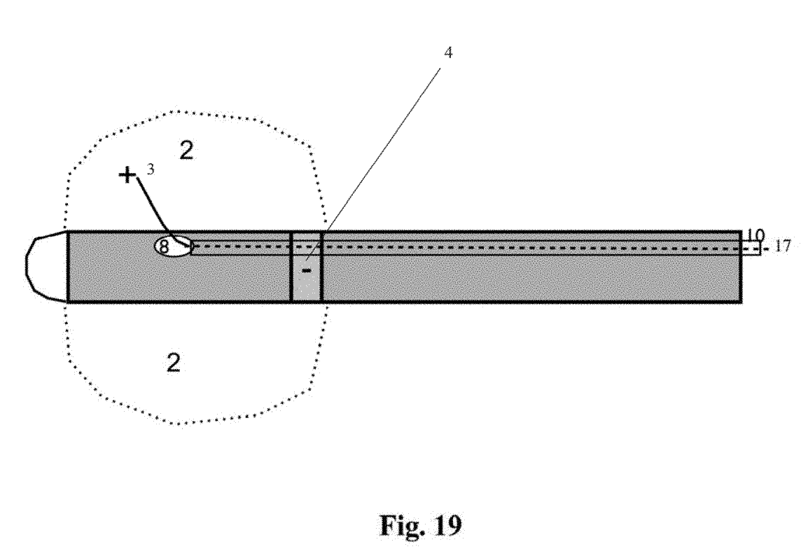

In an alternative embodiment of EMB treatment probes 20, one of either the positive (+) 3 or negative (−) 4 electrodes is on an outer surface of EMB treatment probe 20, while the other polarity of electrode is placed on the tip of a curved needle 17 inserted through a lumen 10 in the interior of core 21. Except for active surface 25 and a side hole 8, through which needle 17 may exit lumen 10, insulating sheath 23 may completely envelope probe 20 to isolate the two electrodes (see FIG. 19).

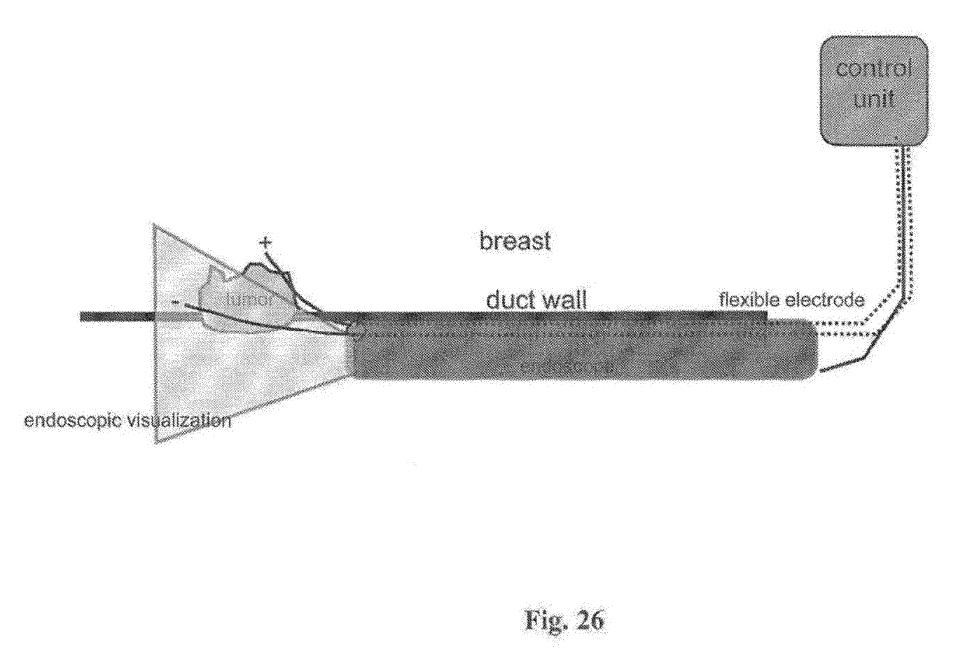

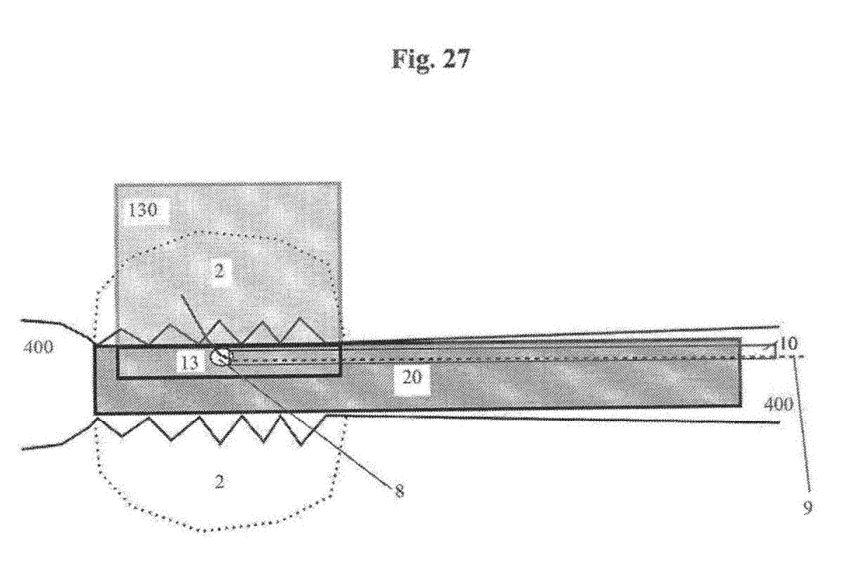

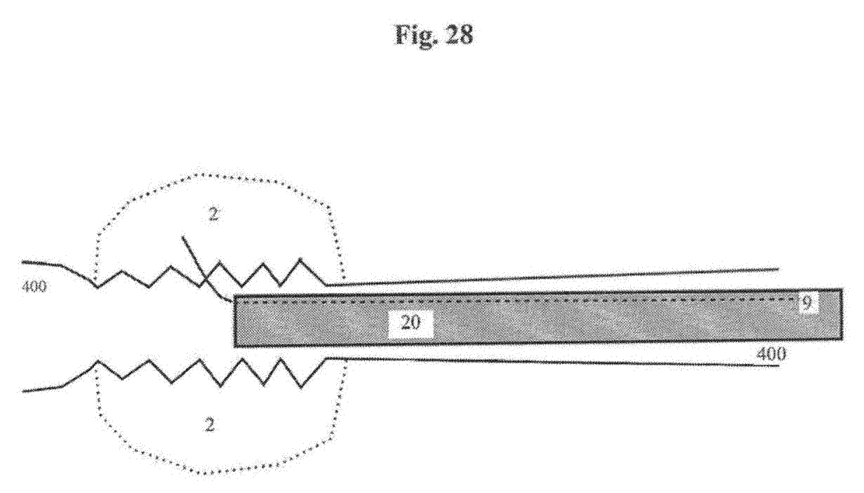

In yet another embodiment, the two curved needle electrodes can be placed through a scope and visualized as they extend out of the scope. For example, in the treatment of breast cancer, the two curved needle electrodes may, under direct scope visualization, pierce the walls of the breast duct and extend into the breast tissue (See FIG. 26).

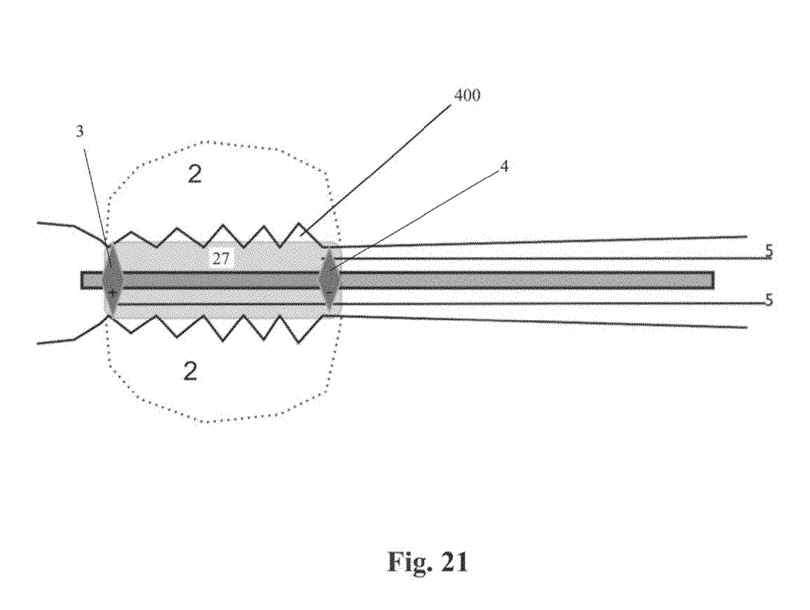

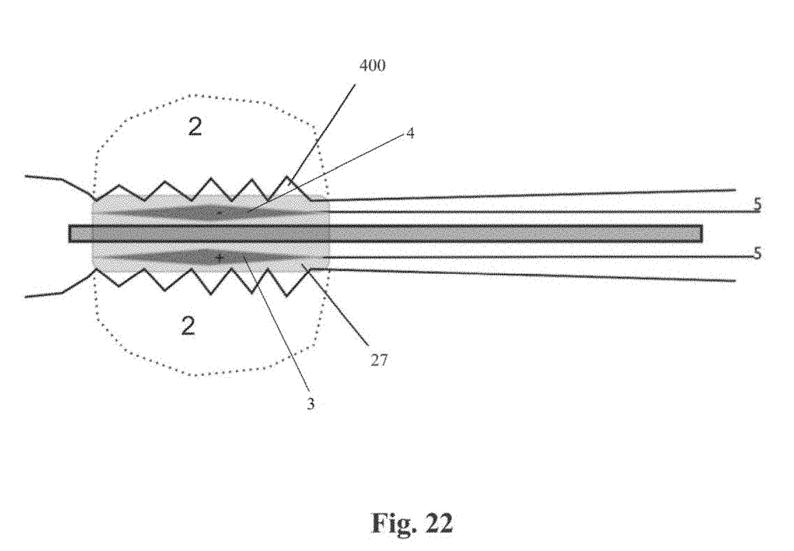

In yet another alternative embodiment of EMB treatment probes 20, unipolar or bipolar electrodes are placed on an expandable balloon 27, the inflation of which may be controlled by the SHCU via a pneumatic motor or air pump, etc. In this embodiment, when the balloon 27 is placed inside a cavity 400 in the human body (proximate a designated treatment area) and inflated, the electrodes on the balloon's surface are forced against the wall of the cavity 400 to provide a path for current to flow between the positive and negative electrodes (see FIG. 21). The positive and negative electrodes can have different configurations on the balloon 27, i.e., they may be arranged horizontally around the circumference of the balloon 27 as in FIG. 21, or longitudinally along the long axis of the balloon as in FIG. 22. In some embodiments, more than one each of positive and negative electrodes may be arranged on a single balloon.

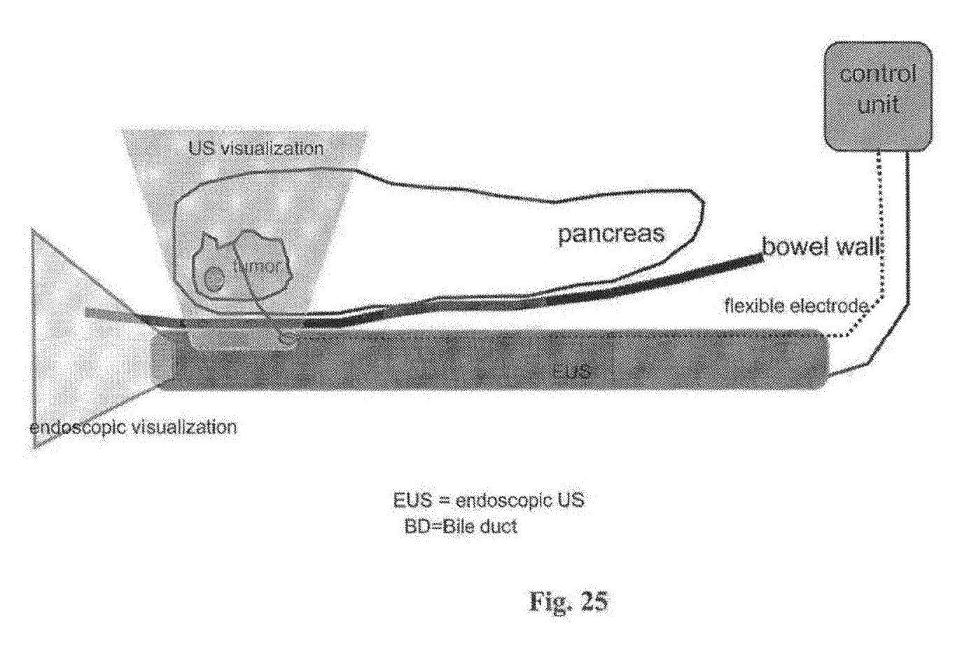

In another embodiment, depending on the location of the target tissue being treated, the EMB treatment probe(s) 20 can be configured to be delivered endoscopically using endoscopic ultrasound (US) as a guidance method. For example, in the treatment of pancreatic cancer, the probes may be placed through the posterior stomach or duodenal wall with treatment administered to the pancreatic tissue involved by cancer (See FIG. 25).

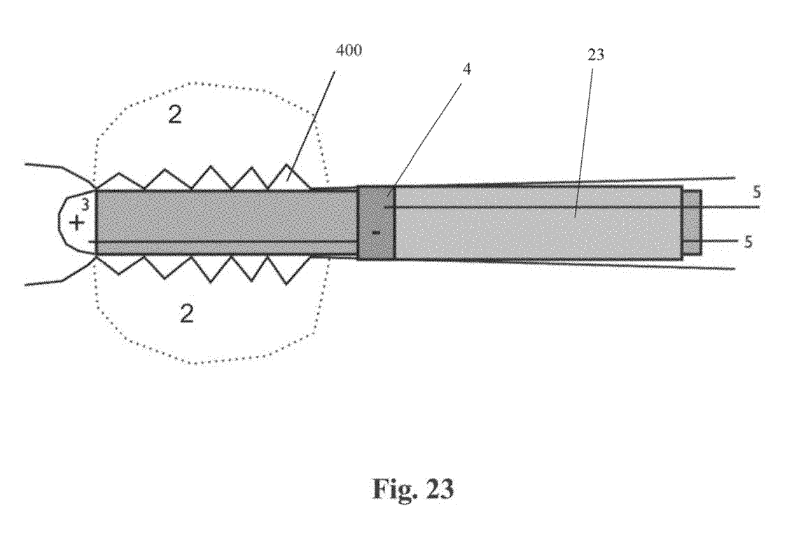

In another embodiment one electrode is on the end of a sheath through which the EMB treatment probe 20 is placed. By moving the catheter various distances from the end of the sheath, various distances between the electrodes can be accomplished thus changing the size and shape of the treatment zone (see FIG. 23).

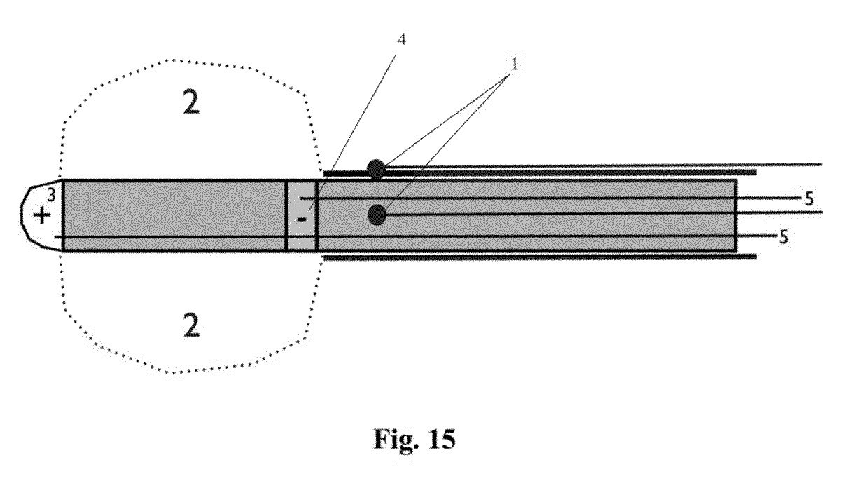

Another embodiment of treatment probe 20 is described with collective reference to FIGS. 15-17, 19-23, 29-30 and 39-40. As shown therein, EMB treatment probes may alternatively be comprised of at least one therapeutic catheter-type probe 20 capable of delivering therapeutic EMB pulsed radio frequency energy or biphasic pulsed electrical energy under sufficient conditions and with sufficient treatment parameters to completely break down the membranes of carcinoma, neoplastic cells or other unwanted tissue. Probes 20 are preferably of the catheter type known in the art and having one or more central lumens to, among other things, allow probe 20 to be placed over a guide wire for ease of insertion and/or placement of probe 20 within a cavity 400 of the human body according to the Seldinger technique. A catheter for this purpose may be a Foley-type catheter, sized between 10 French to 20 French and made of silicone, latex or any other biocompatible, flexible material.

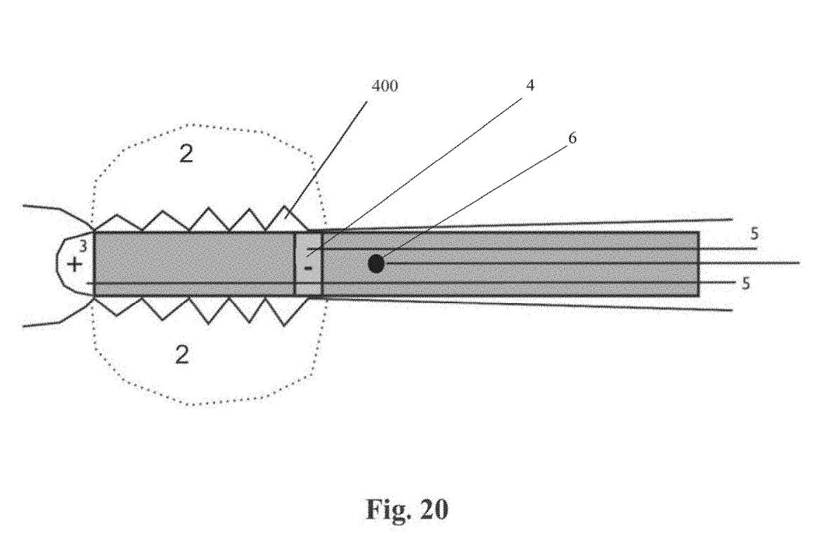

In one embodiment, illustrated in FIG. 20, probe 20 further comprises one positive 3 and one negative 4 electrode disposed on an outer surface of probe 20 and spaced apart by a distance along the longitudinal axis of probe 20 such that current sufficient to deliver the EMB pulses described herein may be generated between the electrodes 3, 4. The spacing between positive 3 and negative 4 electrodes may vary by design preference, wherein a larger distance between electrodes 3, 4 provides a larger treatment area 2. FIG. 20 depicts electrodes 3, 4 on an outer surface of probe 20; alternatively, electrodes 3, 4 are integral to the surface of probe 20. In yet another embodiment, as shown in FIG. 23, one of electrodes 3, 4 (negative electrode 4 as shown in FIG. 23) may be placed on the end of an insulated sheath 23 that either partially or fully surrounds probe 20 along a radial axis thereof and is movable along a longitudinal axis of probe 20 relative to the tip thereof (on which positive electrode 3 is located as shown in FIG. 23) to provide even further customizability with respect to the distance between electrodes 3, 4 and thus the size of treatment area 2. As above, insulating sheath 23 is preferably made of an inert material compatible with bodily tissue, such as Teflon® or Mylar®. Means for enabling the relative movement between probe 20 and insulating sheath 23 include attaching insulating sheath 23 to a fixed member (i.e., a handle) at a distal end of probe 20 opposite the tip of probe 20 by a screw mechanism, the turning of which would advance and retract the insulating sheath 23 along the body of the probe 20. Other means for achieving this functionality of EMB treatment probe 20 are known in the art.

Without limitation, electrodes may be flat (i.e., formed on only a single side of probe 20), cylindrical and surrounding probe 20 around an axis thereof, etc. Electrodes 3, 4 are made of an electrically conductive material. Electrodes 3, 4 may be operatively connected to EMB pulse generator 16 via one or more insulated wires 5 for the delivery of EMB pulses from generator 16 to the treatment area 2. Connection wires 5 may either be intraluminal to the catheter probe 20 or extra-luminal on the surface of catheter probe 20.

In certain embodiments, as shown in FIG. 20, probe 20 further comprises an electromagnetic (EM) sensor/transmitter 6 of the type described above. EM sensors 26 may be located on both probe 20 and optional insulating sheath 23 to send information to the Software Hardware Controller Unit (SHCU) (or other imaging device) for determining the positions and/or relative positions of these two elements and thus the size of the treatment area, preferably in real time. Alternatively, instead of utilizing EM sensors, EMB treatment probes 20 may be tracked in real time and guided using endoscopy, ultrasound or other imaging means known in the art.

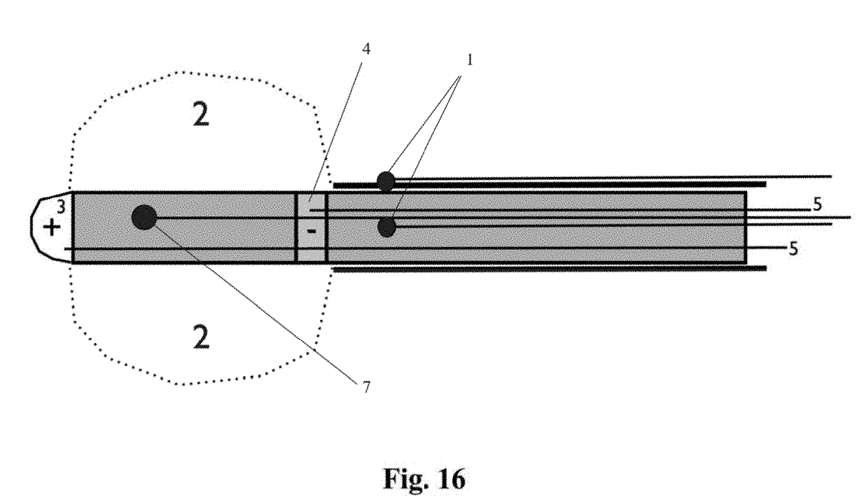

In a preferred embodiment, as shown in FIG. 16, probe 20 further comprises a thermocouple 7 of the type described above on the insulating surface thereof such that the temperature at the wall of the catheter can be monitored and the energy delivery to electrodes 3, 4 modified to maintain a desired temperature at the wall of the probe 20 as described in further detail above.

In yet another alternative embodiment of EMB treatment probes 20, unipolar or bipolar electrodes are placed on an expandable balloon 27, the inflation of which may be controlled by the SHCU via a pneumatic motor or air pump, etc. In this embodiment, when the balloon 27 is placed inside a cavity 400 in the human body (proximate a designated treatment area) and inflated, the electrodes on the balloon's surface are forced against the wall of the cavity 400 to provide a path for current to flow between the positive and negative electrodes (see FIG. 21). The positive and negative electrodes can have different configurations on the balloon 27, i.e., they may be arranged horizontally around the circumference of the balloon 27 as in FIG. 21, or longitudinally along the long axis of the balloon as in FIG. 22. In some embodiments, more than one each of positive and negative electrodes may be arranged on a single balloon.

In certain embodiments of the present invention, the EMB treatment probe 20 is inserted into the treatment area through a body cavity 400, such as the urethra for treatment of a cancerous mass 2 proximate the peri-urethral prostatic tissue. Optionally, the catheter may comprise a non-electrode-containing balloon that is otherwise of the general type described above on its distal end, such that when the balloon (not shown) is inflated, the catheter and EMB treatment probe 20 are anchored within the treatment area for the target tissue by a friction fit of the balloon within the body cavity 400.

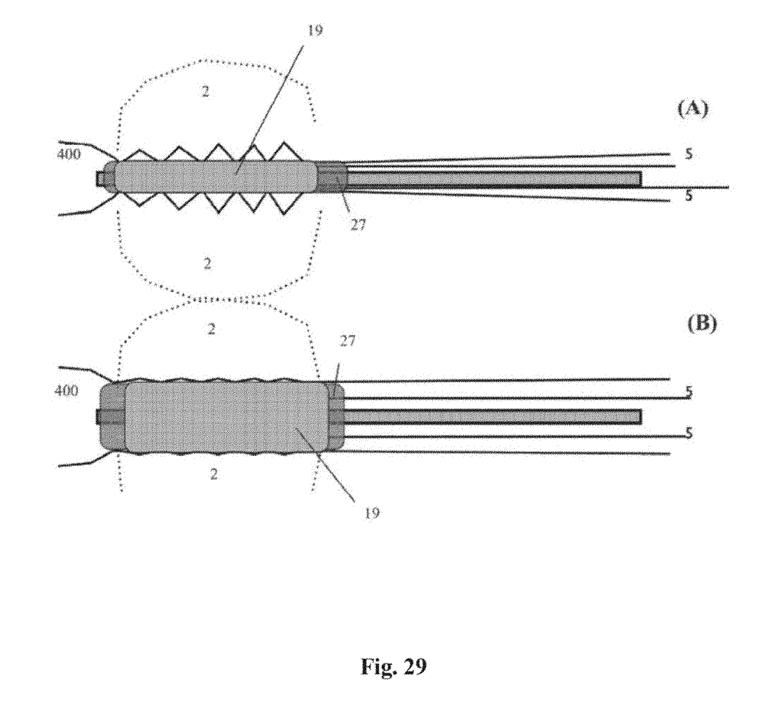

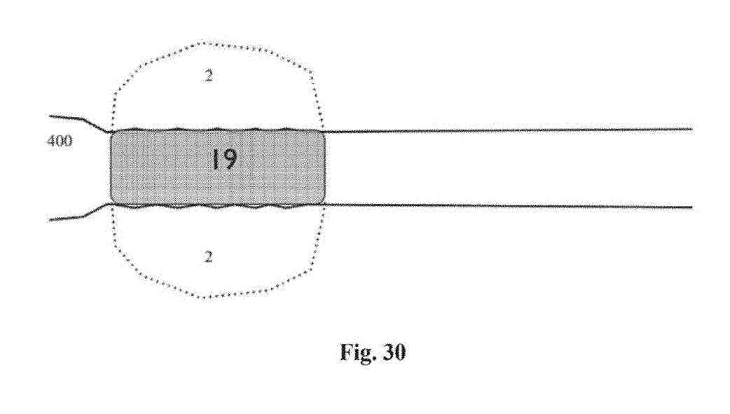

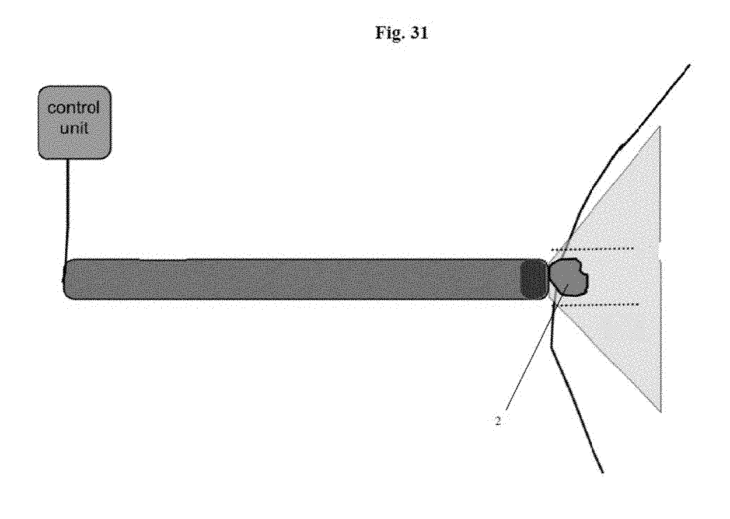

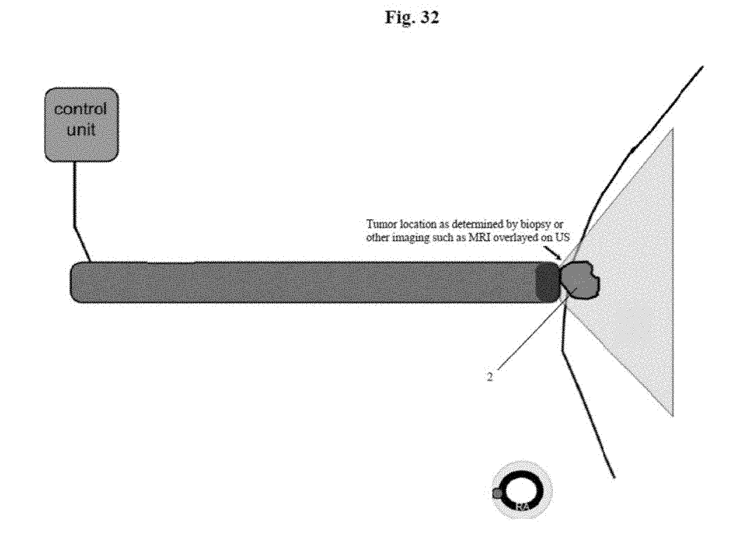

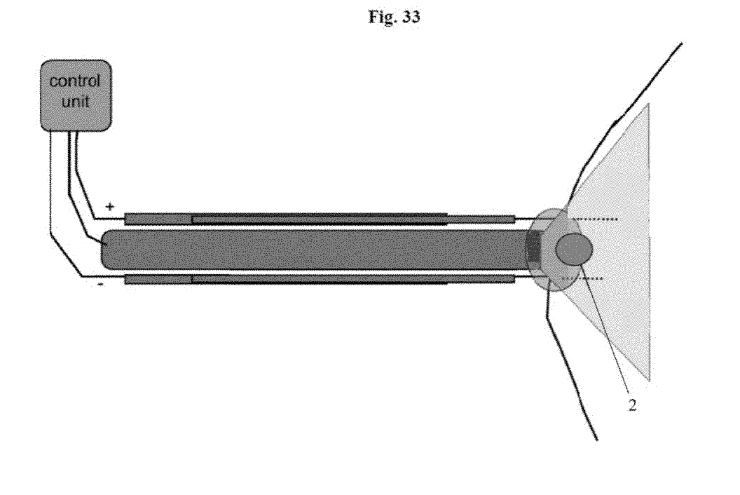

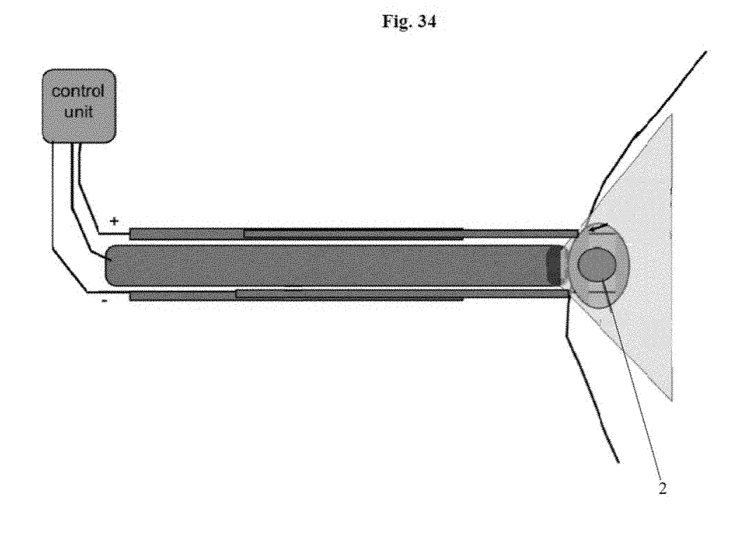

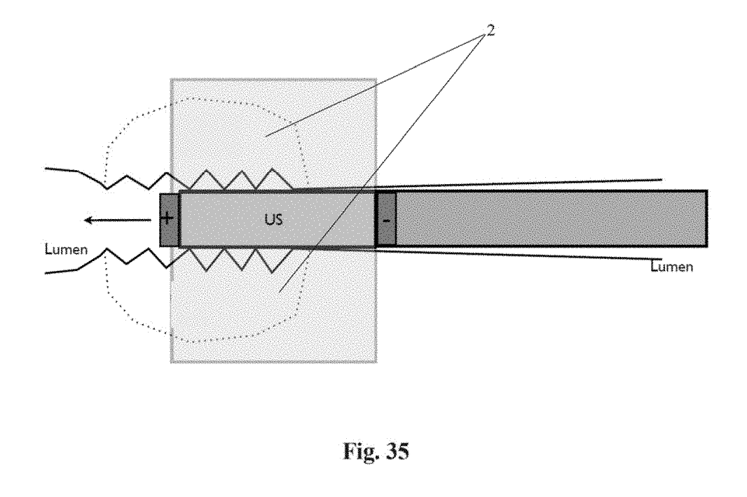

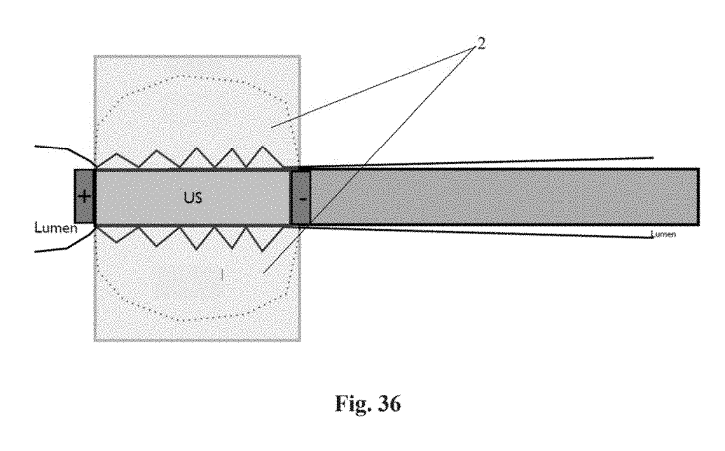

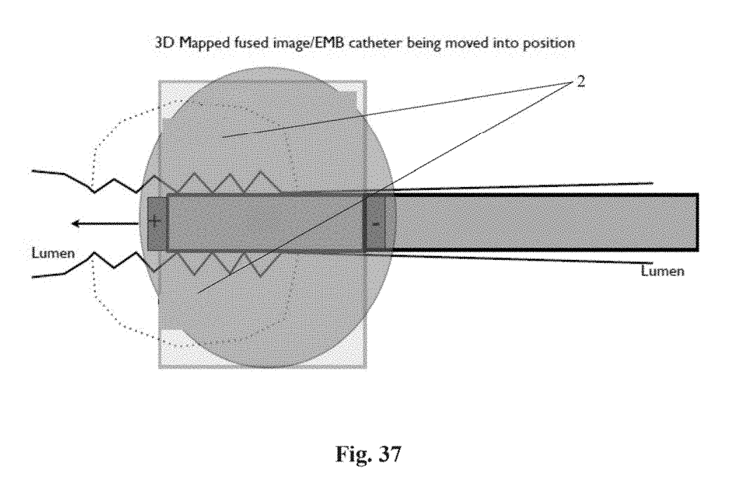

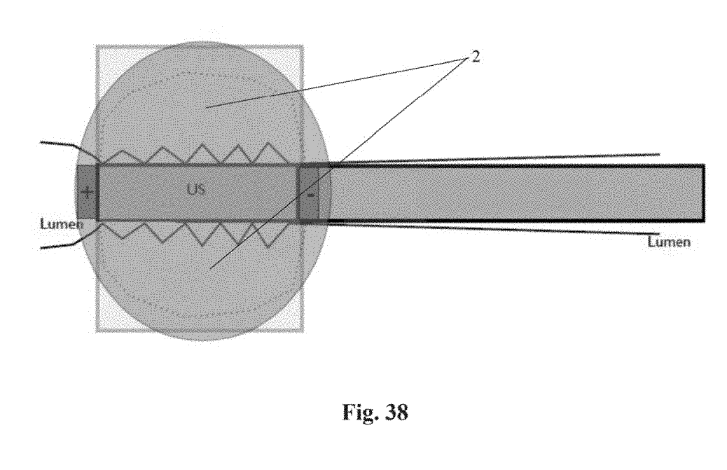

In yet another embodiment, EMB catheter-type probe 20 could deliver a stent 19 to the abnormal region/treatment area 2 in, i.e., the bile or pancreatic duct, which is associated with a narrowing causing obstruction. This configuration would allow the delivery of an EMB treatment protocol at the same time as stent 19 is used to expand a stricture in a lumen. Stent 19 may also comprise conducting and non-conducting areas which correspond to the unipolar or bipolar electrodes on EMB probe 20. An example treatment protocol would include placement of EMB probe 20 having balloon 27 with a stent 19 over the balloon 27 in its non-expanded state (FIG. 29(A)), expansion of balloon 27 which in turn expands stent 19 (FIG. 29(B)), delivery of the RFEMB treatment, and removal of the EMB treatment probe 20 and balloon 27, leaving stent 19 in place in the patient (see FIG. 30).

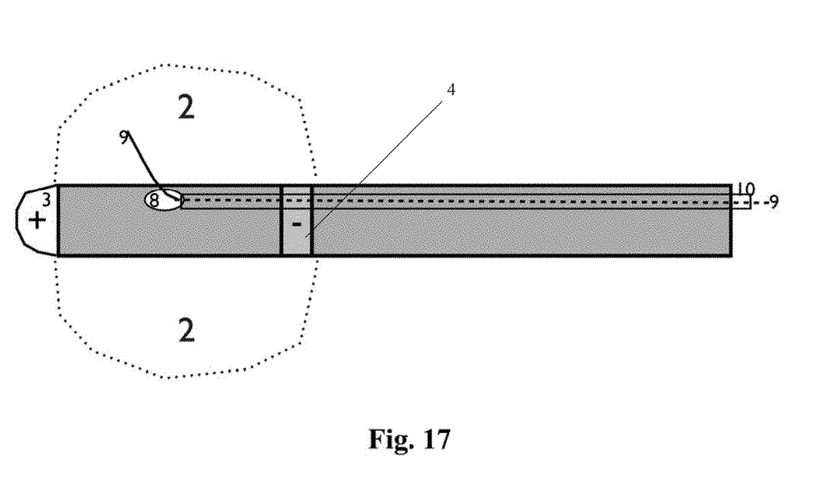

In an alternative embodiment of EMB treatment probes 20, one of either the positive (+) 3 or negative (−) 4 electrodes is on an outer surface of EMB treatment probe 20, while the other polarity of electrode is placed on the tip of a curved needle 9 inserted through an interior lumen 10 such as that described above (see FIG. 17).

In other embodiments, EMB treatment probe 20 is made contiguous with and/or held within a catheter, such as a Foley-type catheter as described above, for ease of insertion of EMB probe 20 into the treatment area. Alternatively, a catheter through which EMB probes 20 are inserted may serve as one of a pair of bipolar electrodes, while the EMB treatment probe 20 is placed directly within the target tissue to serve as the other electrode.

In some embodiments of the present invention, EMB treatment probes 20 contain sensors of the type described by Laufer et al. in “Tissue Characterization Using Electrical Impedance Spectroscopy Data: A Linear Algebra Approach”, Physiol. Meas. 33 (2012) 997-1013, to investigate tissue characteristics to determine cancerous from non-cancerous tissue. Alternatively, or in addition to sensors of the type described by Laufer, EMB treatment catheter type probes 20 may contain sensors to determine cellular content spillage as necessary to quantify cell death or treatment efficacy in the treatment area via EMB; one example of such a sensor is described by Miller et al. in “Integrated Carbon Fiber Electrodes Within Hollow Polymer Microneedles For Transdermal Electrochemical Sensing”, Biomicrofluidics. 2011 Mar. 30; 5(1):13415. The sensors described herein may be placed anywhere on the EMB treatment probes, or inside a lumen therein.

Electrical membrane breakdown, unlike IRE or other thermal treatment techniques, causes immediate spillage of intracellular components of the ruptured cells into an extracellular space and exposes the internal constituent parts of the cell membrane to the extracellular space. The intracellular components include cellular antigens and the internal constituent parts of the cell membrane include antigens specific to the cell membrane which induce an immunologic response to destroy and remove this and like material in the body of the subject. Like material may be other material in the body of the subject having the same cellular antigens or cell membrane specific antigens at locations remote from the treatment site including metastatic tissue. The immunologic response can be enhanced by administration of one or more drugs, materials or agents that increase the immunologic response process including drugs which block the transmission of inhibitory signals to CTLA-4 or PD-1 on activated T lymphocytes, or that binds to S100A9 and modulating regulatory myeloid cell functions, as further described herein. The immunologic response enhancing drug described herein may be any one of those described by U.S. patent application Ser. No. 16/070,072, the disclosure of which is incorporated herein by reference. Specifically, the immunologic response enhancing drug used according to the present invention may be selected from the group including, but not limited to PD-1 inhibitors (including pembrolizumab (marketed as Keytruda) and nivolumab (marketed as Opdivo), PD-1L inhibitors (including atezolizumab (marketed as Tecentriq), and/or CTLA-4 inhibitors, including ipilimumab (marketed as Yervoy). However, it will be understood that any immune response enhancing drug now on the market, in development or that may be developed in the future may be used for this purpose without departing from the scope and spirit of the instant invention.

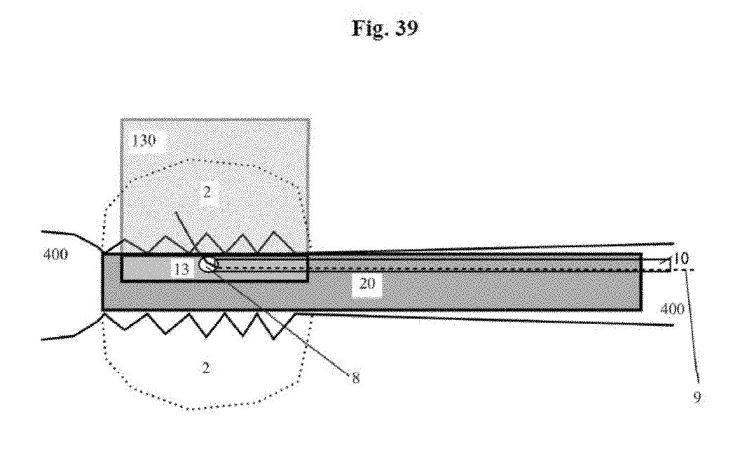

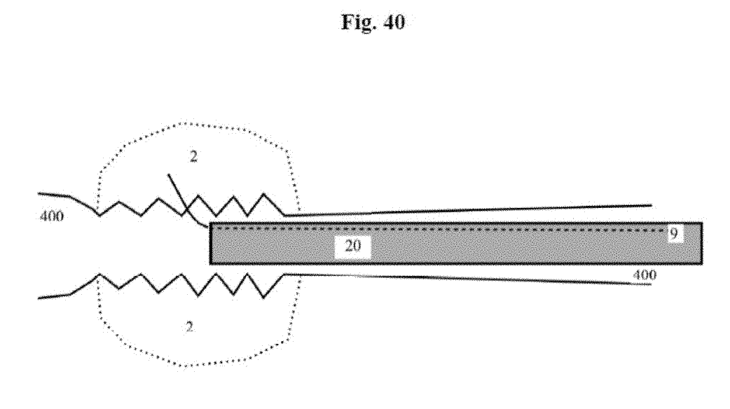

Thus, alternatively or in addition to the sensors described above, EMB treatment probes 20 preferably have a hollow interior defined by an inner lumen 10 (or, in the case of a catheter-type probe, an additional interior lumen 10) of sufficient diameter to accommodate a needle 9 of one or more standard gauges to be inserted there through for the injection of adjuvant immunotherapy type drugs into the lesion formed by EMB treatment to enhance the immunologic response of said treatment (see FIG. 17). A preferred needle may be either a flexible needle, to allow it to be guided through a catheter or one of the catheter-type probes described above, or solid with a fixed curve at the distal end to allow for more precise insertion into the target tissue, or both. Alternatively, the inner lumen 10 may be sized to allow for the injection of biochemical or biophysical nano-materials there through into the EMB lesion to enhance the efficacy of the local tissue destructive effect, or the immunologic response and effect of the EMB treatment, or to allow injection of reparative growth stimulating drugs, chemicals or materials. In one preferred embodiment, as shown in FIG. 17, interior lumen 10 terminates proximate an opening 8 in the side of probe 20 to allow needle 9 to exit probe 20 to access treatment area 2 for delivery of the drugs. In an alternative embodiment, shown in FIG. 40 in the case of a catheter-type probe 20, interior lumen 10 may terminate, and needle 9 may exit, with an opening at distal end of probe 20. In either case, probe 20 may further comprise an ultrasound transducer 13 at a distal end thereof (see FIG. 39) for the formation of an endoscopic viewing area 130 overlapping treatment area 2 to aid in guiding the needle 9 to the appropriate point in the patient for delivery of the drugs. Alternatively, needle 9 may be manipulated via visual guidance. To achieve these functions, needle 9 must be flexible and/or curved to allow it to locate and exit through opening 8 or the distal end of probe 20. Needle 9 is also preferably curved to allow it to pierce the wall of the surrounding tissue, such as bowel, duct or urethra, within the patient's body.

In another embodiment, interior lumen 10 may be sized to allow for the injection of biochemical or biophysical nano-materials there through into the EMB lesion to enhance the efficacy of the local tissue destructive effect, or the immunologic response and effect of the EMB treatment, or to allow injection of reparative growth stimulating drugs, chemicals or materials.

A lumen 10 of the type described herein may also advantageously allow the collection and removal of tissue or intra-cellular components from the treatment area or nearby vicinity. This functionality may take the place of the trackable biopsy needle 200 described in more detail below, and can be used for such purposes before, during or after the application of EMB pulses from the EMB treatment probe 20.

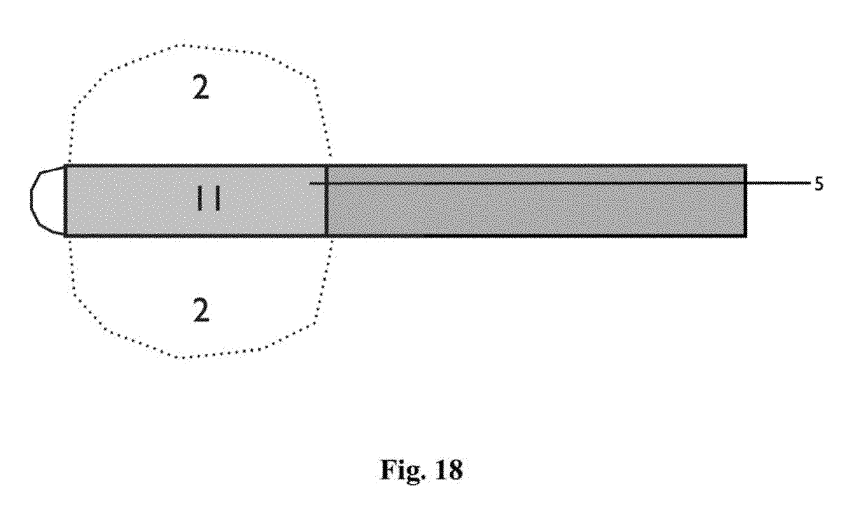

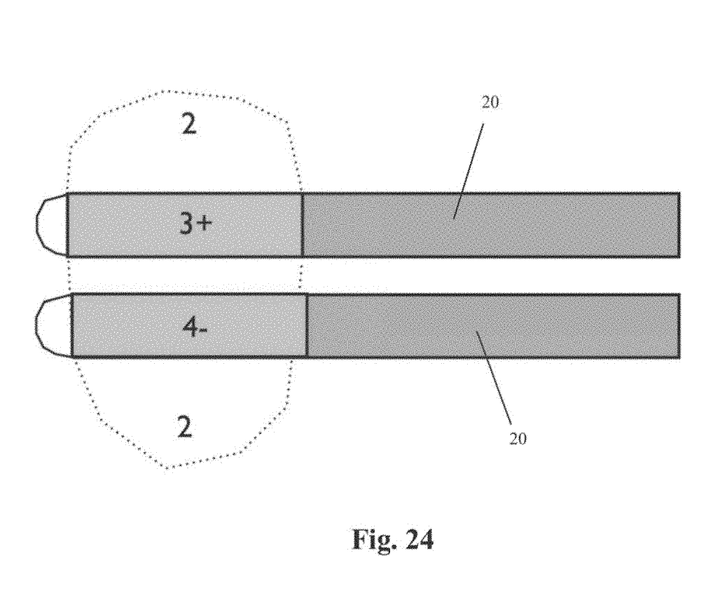

One of ordinary skill in the art will understand that the EMB treatment probe(s) 20 may take various forms provided that they are still capable of delivering EMB pulses from the EMB pulse generator 14 of the type, duration, etc. described above. For example, the EMB treatment probes 20 have been described herein as a rigid assembly, but may also be semi-rigid assembly with formable, pliable and/or deformable components. As another example, EMB treatment probes 20 may be unipolar 11 (see FIG. 18) and used with an indifferent electrode placed on a remote location from the area of treatment (see FIG. 18). In yet another embodiment, two EMB treatment probes 20 may be used, wherein each probe has one each of a positive and negative electrode (See FIG. 24).

Also as described herein, in preferred embodiments, the inventive system also utilizes an optimized cryoablation or cooling pre-cycle to “pre-stress” the cell membrane to increase the efficacy of, and reduce the length of treatment under, a subsequent RFEMB protocol. The use of a cryoablation pre-cycle before the application of a RFEMB treatment protocol cools and “hardens” the membrane of the target cells such that the EMB treatment protocol requires a shorter period of energy application in the form of fewer electrical pulses applied to the target tissue. Thus in preferred embodiments, the functionalities of administering both EMB treatment and cryotherapy are incorporated into the same treatment probe, which also preferably includes a means for an injection (preferably at a constant volume) of immunologic response enhancing drugs such as one or more of those described herein immediately after or in close proximity to the end of the cryo/EMB treatment protocol.

Exemplary devices that may be used for this purpose are illustrated in FIGS. 41-48. Referring to FIG. 41, an injection device 100 is part of a system 101 that is capable of administering both extreme cold as well as electric pulses to tissues and/or tumors. The injection device 100 has two electrode cryoprobes, including a positively-charged cryoprobe 110 and a negatively-charged cryoprobe 130. Each cryoprobe 110, 130 is a generally cylindrical probe that is inserted into a target tissue 102 at a first end 111, 131 and grasped by a user at a second end 112, 132. Each cryoprobe 110, 130 can be individually manipulated by a user. Alternatively, both cryoprobes 110, 130 can be contained within a larger housing (not shown for clarity) that permits the user to insert both cryoprobes 110, 130 into the target tissue 102 simultaneously at a known distance from each other. In some embodiments, the two cryoprobes 110, 130 contained within a housing can be arranged such that the distance separating the two cryoprobe electrodes 110, 130 can be increased or decreased by the user.

Each cryoprobe 110, 130 has a central gas supply cannula 114, 134 running from the first ends 111, 131 to the second ends 112, 132 of the cryoprobes 110, 130. Each central gas supply cannula 114, 134 is attached at the second end 112, 132 of each probe to a cryomachine 190. The cryomachine 190 serves as a source of cooled gas that is pumped via gas supply lines 192 to enter the central gas supply cannulas 114, 134 at the second ends 112, 132 of the cryoprobes and be delivered to cooling heads 116, 136 at the first ends 111, 131 of the cryoprobes and thereby to the tissue 102. The cooling heads 116, 136 are configured to pierce and be inserted into the tissue 102 as is known in the art, and can be flat or pointed in shape. The cooling heads 116, 136 are generally made of metal or other material that has a high conductance so as to allow the cold gas entering the cooling heads 116, 136 via the central gas supply cannulas 114, 134 to thermally interact with the tissue 102.

Gas return channels 118, 138 concentrically surround the central gas supply cannulas 114, 134 and are fluidly connected to the cannulas such that cooled gas enters the cooling heads 116, 136 and then flows back through the gas return channels 118, 138 to return to the cryomachine 190 via gas return lines 194. Layers of thermal insulation 120, 140 protect the user grasping the cryoprobes 110, 130 from the cold gas running through the gas return channels 118, 138. Layers of electrical insulation 122, 142 and the layers of thermal insulation 120, 140 concentrically surround the outer surfaces of the gas return channels 118, 138. The layers of electrical insulation 122, 142 protect the user and electrically isolate the body of each cryoprobe 110, 130 from electrical pulses generated by an electrical pulse generator 180. The order of layers of electrical insulation 122, 142, thermal insulation 120, 140 and the outer surfaces of the gas return channels 118, 138 may be placed in differing orders.

The electrical pulse generator 180 is connected by wires 182 to the second ends 112, 132 of the cryoprobes 110, 130 such that electrical pulses are transmitted to the cooling heads 116, 136 and in turn administered to the tissue 102. The cooling heads 116, 136 therefore serve the dual function of administering cold as well as the electrical impulses to the target tissue 102. The electrical pulses can be transmitted along the length of the cryoprobes 110, 130 via wires layered between the layers of electrical insulation 122, 142 and the layers of thermal insulation 120, 140. In some embodiments, at least a portion of the gas return channels 118, 138 are electrically conductive and also serve the function of transmitting the electrical pulses to the tissue 102 via the cooling heads 116, 136.

The electrical pulse generator 180 is arranged to generate a positive charge via the positively-charged cryoprobe 110 and a negative charge via the negatively charged cryoprobe 130. The injection device 100 is therefore capable of delivering electrical pulses as well as cold temperatures to the target tissue 102. For simplicity, the positively-charged cryoprobe 110 and the negatively-charged cryoprobe 130 can be identical in structure. The two cryoprobes 110, 130 are inserted into the target tissue 102 at a desired distance of separation from each other (e.g., 2 mm, 5 mm, 10 mm), thereby creating a cryolesion zone 104 that surrounds and extends between the tips of the cryoprobes 110, 130. This arrangement of the two cryoprobes 110, 130 also creates an RE (Reversible Electroporation) zone 106 in relation to the cryolesion zone 104.

The configuration of the cryolesion zone 104 can be varied by the user. In some instances, the cooling heads 116, 136 are retractable into the bodies of the cryoprobes 110, 130, e.g., the length of the cooling heads 116, 136 extending from the end of the thermal insulation layers 120, 140 can be reduced by retracting the cooling heads 116, 136 such that more or all of their surface area is covered by the thermal insulation layers 120, 140. Similarly, the length of the cooling heads 116, 136 extending from the end of the thermal insulation layers 120, 140 can be increased by extending the cooling heads 116, 136 such that less of their surface area is covered by the thermal insulation layers 120, 140. The insulation layers 120, 140 and 122, 144 are repositionable during use of the injection device 100. The user can also modify the temperature of the gas exiting the cryomachine 190 and entering the tissue 102. The configuration of the RE zone 106 can be varied by the user by modulating the electrical pulses exiting the electrical pulse generator 180. The variables can be altered such that the cryolesion zone 104 is smaller than, the same size as, or larger than the RE zone 106.

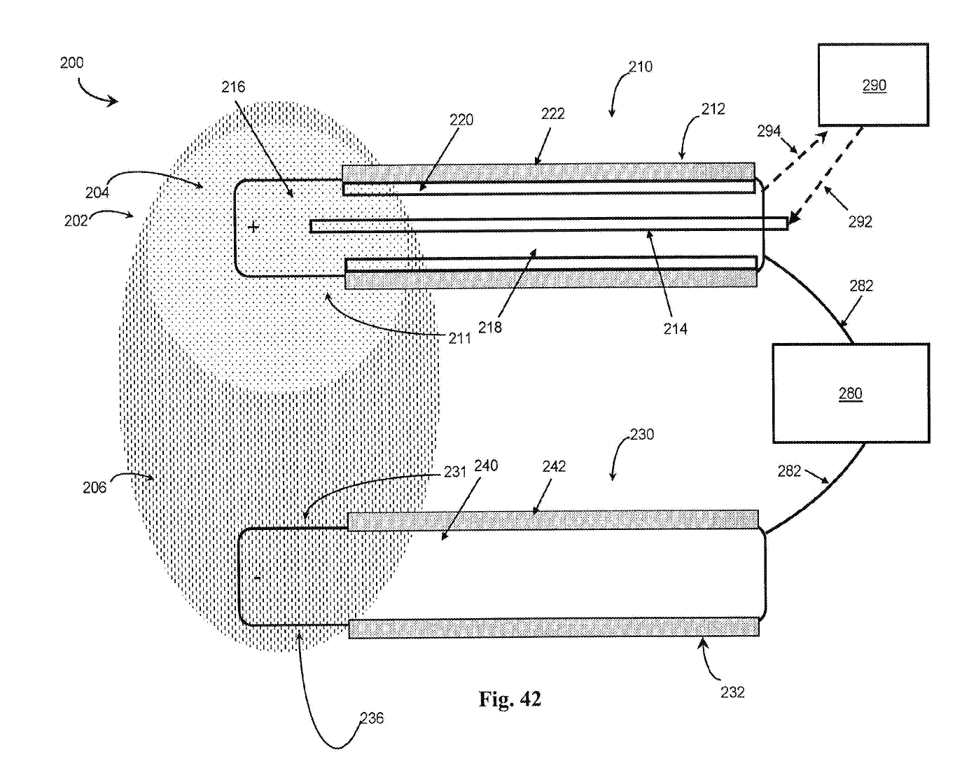

Referring to FIG. 42, an additional embodiment of an injection device 200 that is capable of delivering both cold and electrical pulses to a target tissue 202 is shown. Many of the elements of the electrode cryoprobe 200 are identical to those shown in FIG. 41. A positively-charged cryoprobe 210 has a first end 211 and a second end 212, and a central gas supply cannula 214 running from the first end 211 to the second end 212. The central gas supply cannula 214 is attached at the second end 212 to a cryomachine 290 that is a source of cooled gas that is pumped via a gas supply line 292 to enter the central gas supply cannula 214 and be delivered to a cooling head 216 at the first end 211 of the cryoprobe and thus to the tissue 202. The cooling head 216 is configured to pierce and be inserted into the tissue 202 as is known in the art, and can be flat or pointed in shape, and is generally made of metal or other material that has a high conductance.

A gas return channel 218 concentrically surrounds the central gas supply cannula 214 and is fluidly connected to the cannula 214 such that cooled gas enters the cooling head 216 and then flows back through the gas return channel 218 to return to the cryomachine 290 via a gas return line 294. A layer of thermal insulation 220 protects the user grasping the cryoprobe 210 from the cold gas running through the gas return channel 218. A layer of electrical insulation 222 concentrically layers the outer surface of the gas return channel 218 which is also concentrically surrounded by the layer of thermal insulation 220.

An electrical pulse generator 280 is connected by wires 282 to the second end 212 of the cryoprobe 210 and also to the second end 232 of an electric probe 230. The electric probe 230 is similar to cryoprobe 210, having a first end 231 that is insertable into the tissue 202 and a second end 232 that connects to the electrical pulse generator 280. However the electric probe 230 is not connected to the cryomachine 290 and does not have the structure (e.g., a central gas supply cannula, a gas return channel, gas supply and return lines) to administer cryotherapy to the tissue 202. The electric probe 230 has a tissue insertion head 236 that does not cool the tissue 202 but does administer the electric therapy. The electric pulse generator 280 transmits electrical pulses to the cooling head 216 and tissue insertion head 236 and in turn to the tissue 202. The cooling head 216 therefore serves the dual function of administering cold as well as the electrical impulses to the target tissue 202 while the tissue insertion head 236 administers the electrical impulses only. The electrical pulses can be transmitted along the length of the cryoprobe 210 and electric probe 230 via wires attached to layers of electrical insulation 222, 242. In some embodiments, at least a portion of the bodies of the cryoprobe 210 and electric probe 230 are electrically conductive and also serve the function of transmitting the electrical pulses to the tissue 202. The electrical pulse generator 280 is arranged to generate a positive charge via the positively-charged cryoprobe 210 and a negative charge via the negatively-charged electric probe 230.

The cryoprobe 210 and electric probe 230 are inserted into the target tissue 202 at a desired distance of separation from each other (e.g., 2 mm, 5 mm, 10 mm), thereby creating an RE zone 206 that surrounds and extends between the cryoprobe 210 and electric probe 230. As only cryoprobe 210 administers cold to the tissue 202, a created cryolesion zone 204 is smaller than the cryolesion zone 104 created with two cryoprobes and surrounds the first end 211 of the cryoprobe 210.

The configuration of the cryolesion zone 204 can be varied by the user as for cryoprobe injection device 200 by arranging the cooling head 216 to be retractable into the body of the cryoprobes 210. The user can also modify the temperature of the gas exiting the cryomachine and entering the tissue 202. The size of the RE zone 206 can be varied by modulating the electrical pulses exiting the electrical pulse generator 180.

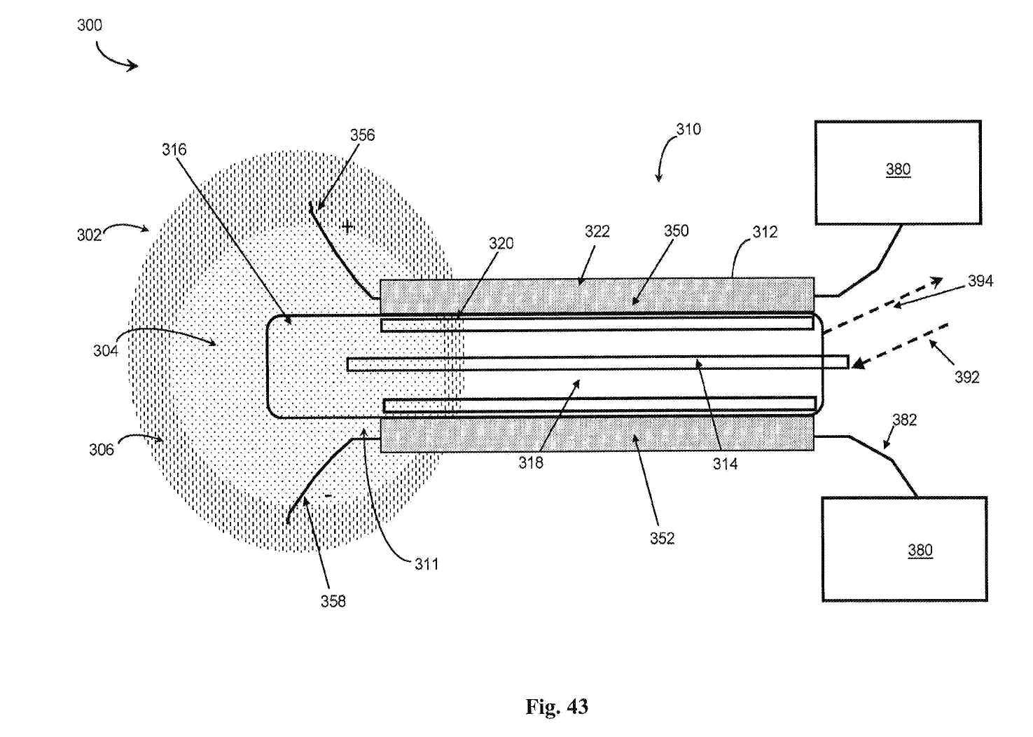

Shown in FIG. 43 is an embodiment of an injection device 300 that has a single cryoprobe 310. The elements of the injection device are similar to the previous embodiments, however the injection device 300 has a single cryoprobe 310. The cryoprobe 310 is capable of delivering both cold and electrical pulses to a target tissue 302 and has a first end 311, a second end 312, a central gas supply cannula 314 running between them and attached to a cryomachine 390 (not shown) that is a source of cooled gas pumped via a gas supply line 392 to the cryoprobe 310 and delivered to a cooling head 316 and removed by a gas return channel 318 concentrically surrounding and fluidly connected to the central gas supply cannula 314. A layer of thermal insulation 320 and a layer of electrical insulation 322 are also present.

One or two electrical pulse generators 380 (as shown in FIG. 43) are connected by wires 382 to the second end 312 of the cryoprobe 310. The wires 382 attach to a pair of wires 350, 352 that terminate in electrodes 356, 358 that exit the body of the cryoprobe and enter the tissue 302 alongside the cooling head 316. The wires 350, 352 are embedded in the electrical insulation layer 322, e.g., by piercing the electrical insulation layer 322 or by insertion into channels that run the length of the electrical insulation layer 322. The wires 350, 352 and electrodes 356, 358 can attach to each other, respectively, or in some embodiments the positive wire 350 and positive electrode 356 are the same continuous wire and the negative wire 352 and negative electrode 358 are the same continuous wire.

The electrodes 356, 358 are shaped such that when extended into the tissue 302 the electrodes curve away from the body of the cryoprobe 310. When retracted, the electrodes 356, 358 are held in a linear shape to better align with the body of the cryoprobe. The electrodes 356, 358 can be formed of e.g., nickel titanium (also known as nitinol). The curvature of the electrodes 356, 358 allows the user to extend the resulting RE zone 306 beyond the cryolesion zone 304. The user can extend the electrodes 356, 358 and transmit electric pulses before, during, or after the cryotherapy treatment.

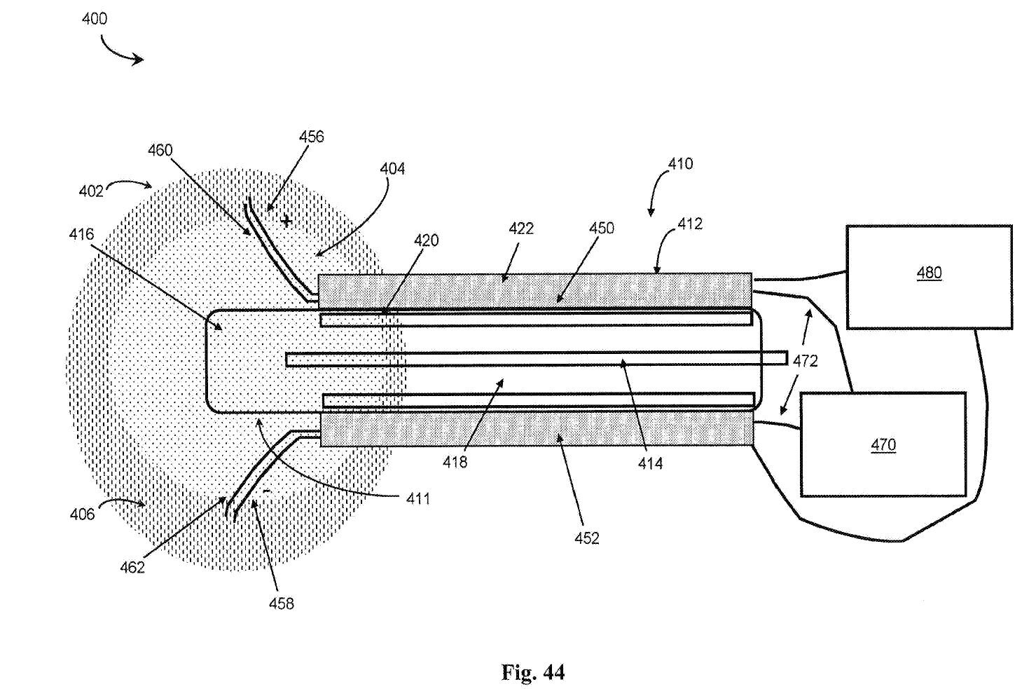

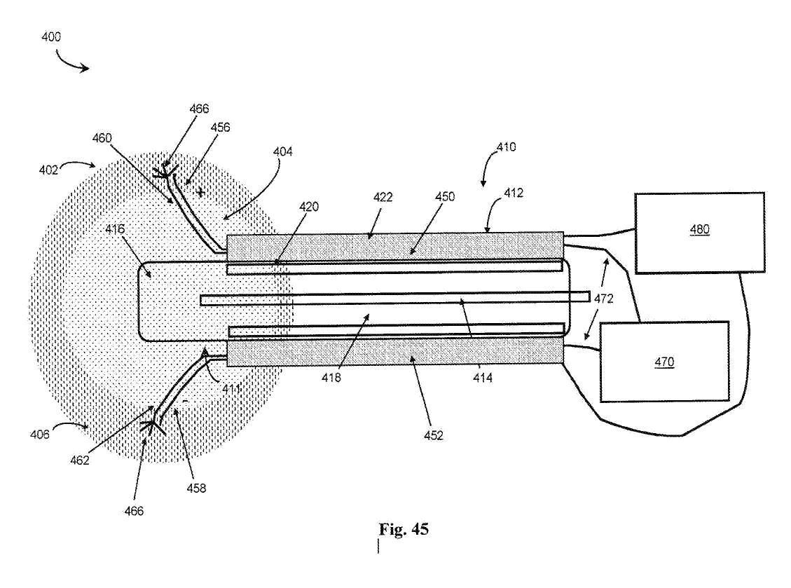

FIG. 44 shows an injection device 400 similar to that of FIG. 43 (with reference labels referring to the same elements as in FIG. 43 but raised by 100). However injection device 400 is capable of injecting plasmids into tissue 402 as well as administering electrotherapy and cryotherapy. The cryoprobe 410 has needles 460, 462 that extend approximately parallel with electrodes 456, 458 and are inserted into tissue 402. At the second end 412 of the cryoprobe, the needles 460, 462 are fluidly connected to tubes 472 which receives fluid from a fluid reservoir 470. For example, the fluid reservoir 470 can be a syringe. Fluid, e.g., plasmids, inside the fluid reservoir 470 can therefore be administered to the tissue 402. The needles 460, 462 are fully or partially retractable into the body of the cryoprobe 410 as are the electrodes 456, 458. The needles 460, 462 and electrodes 456, 458 can be retracted simultaneously or independently of each other. The needles 460, 462 are also repositionable within the tissue 402. In some embodiments, shown in FIG. 45, the needles 460, 462 can have multiple tines 466. Multiple tines 466 can allow the user greater control over the spread and distribution of the injected materials or medications in a more quickly and precisely controllable pattern and at a specific distance from the central probe.

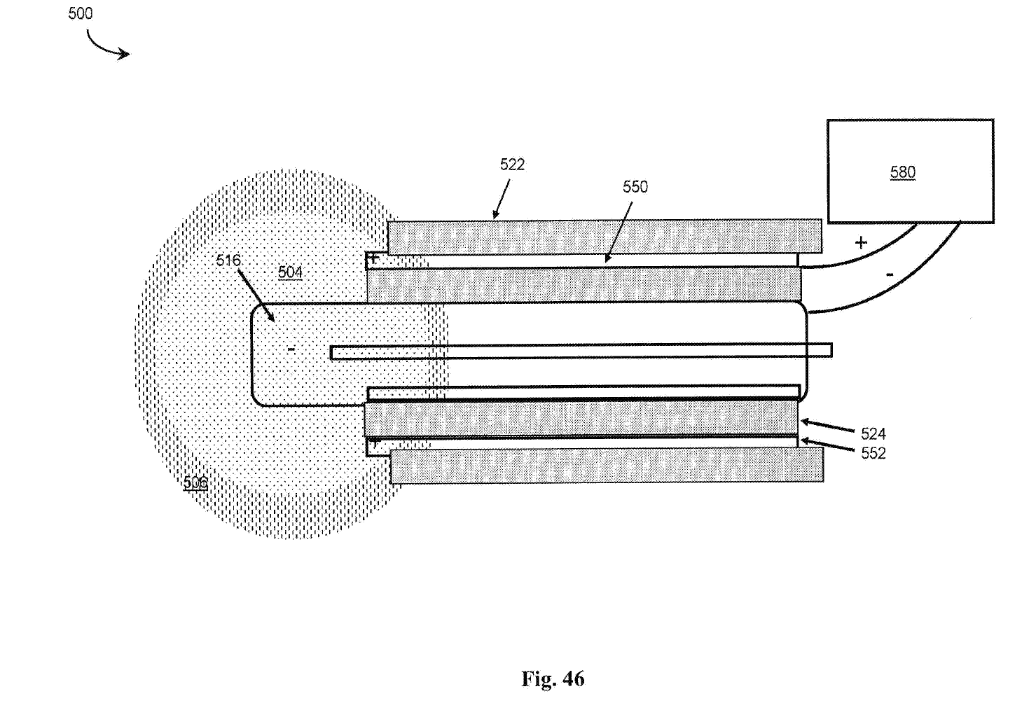

FIG. 46 shows a cryoprobe 500 with two layers of electrical insulation 522, 524. Wires or electrical conduits 550, 552 are sandwiched between the two layers of electrical insulation 522, 524 and carry positive charge from the electric pulse generator 580. The body of the cryoprobe 500 terminates in the cooling head 516 and acts as an electrical conduit for the negative charge generated by the electrical pulse generator 580. Each of the two layers of electrical insulation 522, 524 is independently positionable and retractable.

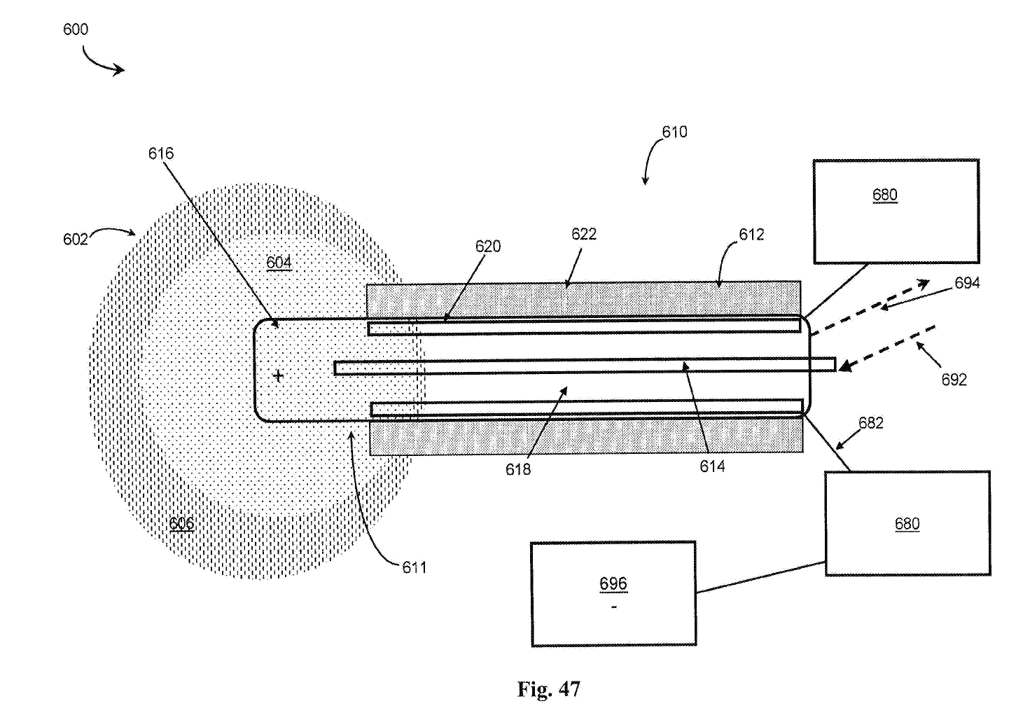

Shown in FIG. 47 is an embodiment of an injection device 600 that has a single cryoprobe 610. The elements of the injection device are similar to the previous embodiments, however the injection device 600 has a single cryoprobe 610 that works with an indifferent electrode 696, which is a remote electrode placed either upon a single limb or connected with the central terminal and paired with an exploring electrode of cryoprobe 610. The cryoprobe 610 is capable of delivering both cold and electrical pulses to a target tissue 602 and has a first end 611, a second end 612, and a central gas supply cannula 614 running between them and attached to a cryomachine 690 (not shown) that is a source of cooled gas pumped via a gas supply line 692 to the cryoprobe 610 and delivered to a cooling head 616 and removed by a gas return channel 618 concentrically surrounding and fluidly connected to the central gas supply cannula 614. A layer of thermal insulation 620 and a layer of electrical insulation 622 are also present. One or two electrical pulse generators 680 (as shown in FIG. 47) are connected by wires 682 to the second end 612 of the cryoprobe 610, and also to the indifferent electrode 696.

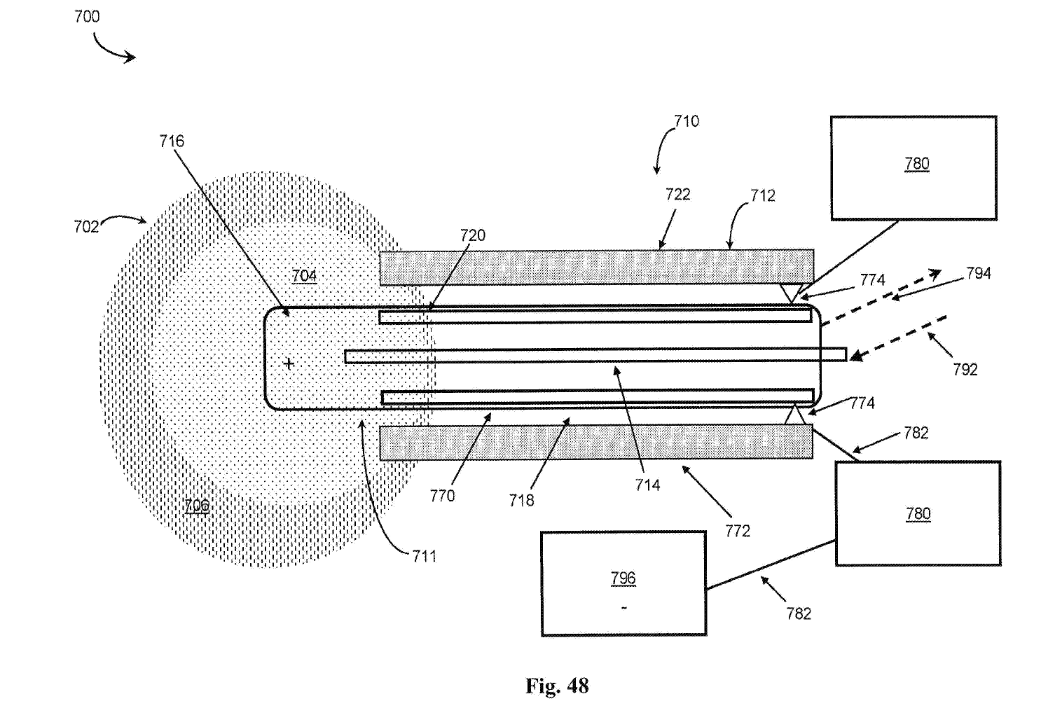

Referring to FIG. 48, an additional embodiment of an injection device 700 is described. The elements of the injection device 700 are similar to the previous embodiments. The injection device 700 has a single probe 710 that can be configured to work with an indifferent electrode 796. In some embodiments the injection device 700 includes a cryoprobe which is capable of delivering both cold and electrical pulses to a target tissue 702, and has a first end 711 and a second end 712.

Probe 710 is made of two different portions, a central portion 770 and concentric portion 772. The central portion has central gas supply cannula 714 running between the first and second ends of the probe 710 and is attached to a source of cooled gas pumped via a gas supply line 692 to the central portion 770 and delivered to a cooling head 716, and removed by a gas return channel 718 concentrically surrounding and fluidly connected to the central gas supply cannula 714. A layer of thermal insulation 720 surrounds the gas channels.

The concentric portion 772 surrounds the central portion 770, and is surrounded by a layer of electrical insulation 722. One or two electrical pulse generators 780 (two are shown in FIG. 48) are connected by wires 782 to the second end 712 of the probe 710, specifically at concentric portion 772, and also to the indifferent electrode 796. The concentric portion 772 is attachable to and removable from the central portion 770. Concentric portion 772 has the form of a sheath that surrounds the internal central portion 772 and the concentric portion 772 can be slid onto and off of the central portion 770 by repositioning the concentric portion 772 relative to the axial length of the central portion 770.

Electrical contacts 774 are included on the concentric portion 772, (e.g., on its inner surface). The electrical contacts 774 bring the wires 782 attached to the electrical pulse generator(s) 780 and indifferent electrode 796 into electric contact with an electrically conducting part of the central portion 770. If the central portion 770 is made of metal, or other conducting material, the electric impulses are thereby transmitted along the body of the central portion to the cooling head 716 to administer the electric therapy to the tissue 702. Alternatively, the central portion 770 can have wires configured to transmit current from the pulse generator(s) along the length of the central portion 770.

The embodiment shown in FIG. 48 is particularly advantageous. The concentric portion 772 can be manufactured separately from the central portion 770. For example, central portion 770 can be a complete cryoprobe that is traditionally used in such therapies. Attaching the concentric portion 772 to the outside of the central portion 770 increases the functionality of the probe, allowing the previously single-use cryoprobe to additionally provide electric RF-EMB treatment capability.

The embodiment of FIG. 48 allows a user to perform combined electric RF-EMB treatment and cryotherapy in a highly precise manner, and with increased flexibility. The probe 710 can be inserted into the tumor or target tissue 702 as desired. Only the concentric portion 722, the central portion 772, or both the portions and be positioned as desired. In one embodiment, the user inserts the probe 710 with other inner and outer portions, and performs the desired therapeutic protocol. The user then can remove the central portion 770 from the tissue 702 by sliding it out of the concentric portion 772 while the concentric portion 772 remains in place. The user then can replace the removed central portion with a different central portion (e.g., a needle for delivering plasmids as described above, a tool that has neither cryo nor electricity-delivering capability such as a measurement tool, an acidity sensing or bioactive device, a tissue collection tool, a biopsy tool, or a hypothermia probe). The concentric portion 772 remaining in place allows the user to insert the new central portion with high accuracy, precisely returning to the previous location of the first end of the central portion 772 before it was removed from the tissue 702.

In some embodiments, the concentric portion 772 of the probe 710 can be used in conjunction with tools other than a probe inserted within the concentric portion 772. Once in place, the concentric portion 772 acts as a guidance device so that a different tool is inserted into the precise same location with the benefit of the next tool being placed in the same location as the prior tool. The replacement inner tool can be any tool that fits within concentric portion 772 (such a measurement tool). The replacement tool can be energized through the electric contacts 774 on the concentric portion 772.

In some embodiments, tools that replace the inner portion to work with concentric portion 772 can be tools that have corresponding electrical contacts on the body of the tool to mate with the electric contacts 774 on the centric probe portion 772. Such inner tools can be previously existing tools that are modified to have such electrical contacts, or tools designed to include such contacts. Additionally, each tool function can be used to cause a desired effect in the tissue 702, and depending on the characteristic of the replacement inner tool and the parameters used each tool can cause an effect in only a part of the tissue 702.

In some embodiments, probe 710 has a locking mechanism or alignment mechanism between the concentric portion 772 and the central portion 770 (e.g., a lever, spring, clip, or luer-type lock). Once the central portion 770 is inserted into the concentric portion 772, the locking mechanism keeps the inner and outer portions aligned and stationary relative to each other. In some embodiments, the probe 710 will only function once the locking mechanism between the inner and outer portions are engaged. For example, the user would have to twist the central portion 770 into engagement with a ridge on the concentric mechanism, and completing the movement would bring electrical contacts on the central portion into contact with the electrical contacts 774 of the concentric portion.

It will also be understood that, instead of a EMB treatment probe having a lumen capable of providing a delivery path for immunologic response enhancing drugs, such drugs may be administered by any means, including without limitation, intravenously, orally or intramuscularly and may further be injected directly into or adjacent to the target soft tissue immediately before or after applying the EMB electric field. Such immunologic response enhancing drug may be comprised also of autologous dendritic cells.

Injection Needle and Injector

In certain preferred embodiments, a needle used for the injection of an immunologic response enhancing drug is a standalone device not incorporated into an EMB treatment probe as described above with respect to alternative embodiments of the present invention. As will be described, a primary feature of the inventive method is the high-pressure infusion of a immunostimulatory drug formulation into the zone of cancer antigen exposure created by the EMB and/or CRYO/EMB protocol. The needle used for this purpose must be capable of delivering an injectable at a specified flow rate, pressure and/or volume as dictated by the treatment protocol, described herein.

The injection needle used for this purpose is preferably operatively paired with an injector unit for feeding an injectate to the injection needle at the predetermined pressure and/or flow and/or volume parameters. Each of the injection needle and injector unit may, in certain embodiments, be operatively paired with the SHCU to automate, control and/or direct the delivery of the injectate at the specified conditions.

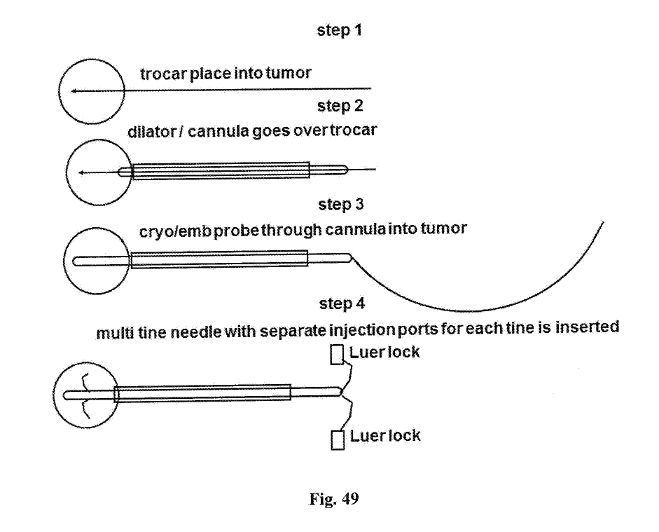

FIG. 49 shows an embodiment according to the present invention whereby a multi-tine needle is used. As shown therein, a multi-tine needle may be sized to fit through a standard-sized dilator or cannula that may be inserted into the target tissue over a standard gauge trocar device as is known in the art. The needle according to the present invention may have at least two tines, each operatively connected to a separate injection port and secured with a Luer lock. A method of using the instant probe may comprise (1) placement of the trocar into the target tissue; (2) sliding the dilator or cannula over the trocar; (3) inserting a cryo/EMB or EMB probe through the cannula into the target tissue for application of the cryoablative and/or EMB treatment as described herein; and (4) removal of the a cryo/EMB or EMB probe and insertion of the multi-tine needle as herein described for application of the anti-tumoral injection.

Trackable Biopsy Needles 200

Unlike irreversible electroporation, electrical membrane breakdown EMB causes immediate visually observable tissue changes which show cellular membrane destruction and immediate cell death. As a result, the method of the present invention may include the biopsy of a portion of the treated target tissue to verify treatment efficacy immediately upon completion of each tissue treatment during the ongoing therapy procedure, while the patient is still in position for additional, continued or further treatment. Alternate uses of such a biopsy needle include confirming accurate placement of the one or more probes used in connection with the treatment described herein, as will be explained.

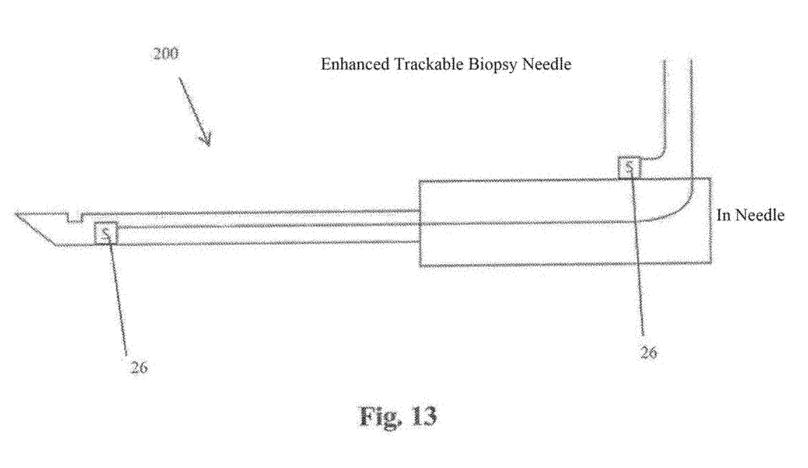

A biopsy needle 200 suitable for this purpose is shown in FIG. 13. Like EMB treatment probes 20, biopsy needle 200 may comprise sensor/transmitters 26 (electromagnetic or otherwise) built into the needle and/or needle body to track the location of the biopsy tip of needle 200 and/or the orientation of the needle 200 as a whole. In certain embodiments, biopsy needle 200 may also comprise sensors to investigate tissue characteristics to determine cancerous from non-cancerous tissue and/or determine cellular content spillage in order to ascertain and/or document cancer cell death, such as those sensors described by Laufer and Miller, above.

Biopsy needle 200 is preferably operatively connected to SHCU 14 to provide real-time data from any sensors contained thereon and to enable real-time tracking of biopsy needle 200 by SHCU 14 to monitor treatment, as described in more detail below. Additional treatment may be immediately administered via, i.e., EMB treatment probe 20, based on the biopsy tissue inspection or result and/or other information obtained from the sensors on biopsy needle 200 or visual determination of treatment efficacy without removing biopsy needle 200 from the treatment area.

Trackable Anesthesia Needles 300

EMB, by virtue of its bipolar wave forms in the described frequency range, does not cause muscle twitching and contraction. Therefore a procedure using the same may be carried out under local anesthesia without the need for general anesthesia and neuromuscular blockade to attempt to induce paralysis during the procedure. Rather, anesthesia can be applied locally for the control of pain without the need for the deeper and riskier levels of sedation.

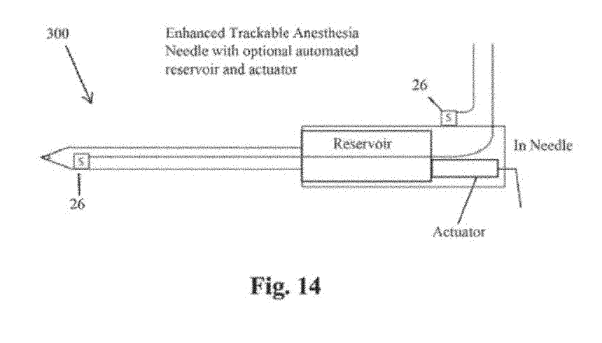

For this purpose, one or more trackable anesthesia needles 300 may be provided. With reference to FIG. 14, Anesthesia needles 300 may be of the type known in the art and capable of delivering anesthesia to the Neurovascular bundles or other potential treatment regions, including the point of entry of needle 300, EMB probe 20, biopsy probe 200 or any of the other devices described herein through the skin to enhance pain relief. Anesthesia needles 300 may also comprise sensor/transmitters 26 (electromagnetic or otherwise) built into the needle and/or needle body to track the location anesthesia needle 300. Anesthesia needles 300 are preferably operatively connected to SHCU 14 to enable real-time tracking of anesthesia needle 300 by SHCU 14 and/or to monitor administration of anesthesia, as described in more detail below.

Alternatively, trackable anesthesia needles 300 may be omitted in favor of conventional anesthesia needles which may be applied by the physician using conventional manual targeting techniques and using the insertion point, insertion path and trajectories generated by the software according to the present invention, as described in further detail below.

Software Hardware Control Unit (SHCU) 14 and Treatment System Software

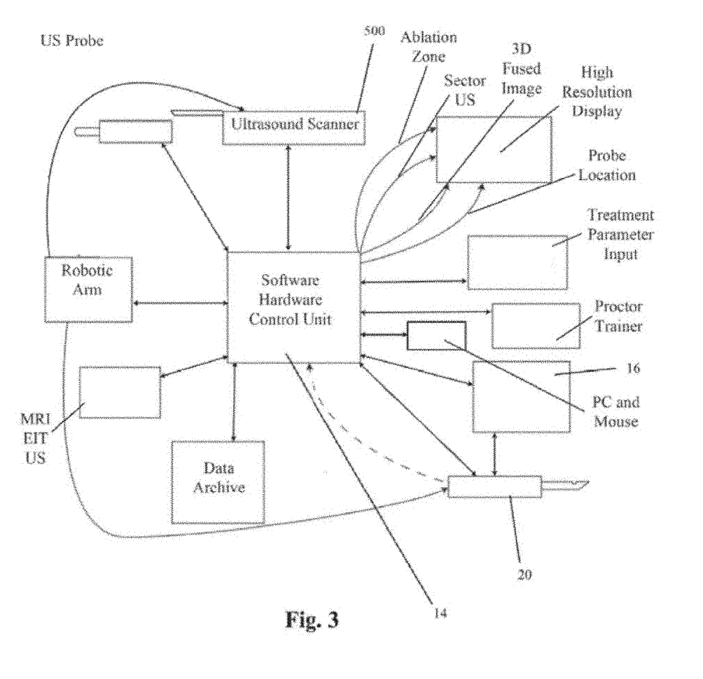

With reference to FIG. 3, in certain embodiments of the instant invention, the Software Hardware Control Unit (SHCU) 14 is operatively connected to one or more (and preferably all) of the therapeutic and/or diagnostic probes/needles, imaging devices and energy sources described herein: namely, in a preferred embodiment, the SHCU 14 is operatively connected to one or more EMB pulse generator(s) 16, EMB treatment probe(s) 20 and/or cryoablation needles and/or cryoablation/EMB needles and/or injection needles, cryomachine(s), trackable biopsy needle(s) 200, fluid pump(s) for controlling the delivery of immune response enhancing drugs as described herein, and trackable anesthesia needle(s) 300 via electrical/manual connections for providing power to the connected devices as necessary and via data connections, wired or wireless, for receiving data transmitted by the various sensors attached to each connected device. SHCU 14 is preferably operatively connected to each of the devices described herein such as to enable SHCU 14 to receive all available data regarding the operation and placement of each of these devices. For example, SHCU 14 may be connected to one or more trackable anesthesia needles 300 via a fluid pump through which liquid medication is provided to anesthesia needle 300 such that SHCU 14 may monitor and/or control the volume, rate, type, etc. of medication provided through needle(s) 300.

In an alternative embodiment, SHCU 14 is also connected to one or more of the devices herein via at least one robot arm such that SHCU 14 may itself direct the placement of various aspects of the device relative to a patient, potentially enabling fully automatized and robotic treatment of certain cancerous or unwanted tissues via the protocols described herein. It is envisioned that the system disclosed herein may be customizable with respect to the level of automation, i.e. the number and scope of components of the herein disclosed method that are performed automatically at the direction of the SHCU 14. At the opposite end of the spectrum from a fully automated system, SHCU 14 may operate software to guide a physician or other operator through a video monitor, audio cues, or some other means, through the steps of the procedure based on the software's determination of the best treatment protocol, such as by directing an operator where to place the EMB treatment probe 20, etc. As examples of semi-automation, SHCU 14 may be operatively connected to at least one robotic arm comprising an alignment tool capable of supporting probe 20, or providing an axis for alignment of probe 20, such that the tip of probe 20 is positioned at the correct point and angle at the surface of the patient's skin to provide a direct path along the longitudinal axis of probe 20 to the preferred location of the tip of probe 20 within the treatment area. In another embodiment, as described in more detail below, SHCU 14 provides audio or visual cues to the operator to indicate whether the insertion path of probe 20 is correct. In each of these variations and embodiments, the system, at the direction of SHCU 14, directs the planning, validation and verification of the Predicted Treatment Zone (to be described in more detail below), to control the application of therapeutic energy and/or cryoablative treatment to the selected region so as to assure proper treatment, to prevent damage to sensitive structures, to enhance the patient's immunologic response to his cancer and/or to provide tracking, storage, transmission and/or retrieval of data describing the treatment applied.

In a preferred embodiment, SHCU is a data processing system comprising at least one application server and at least one workstation comprising a monitor capable of displaying to the operator a still or video image, and at least one input device through which the operator may provide inputs to the system, i.e. via a keyboard/mouse or touch screen, which runs software programmed to control the system in three “modes” of operation, wherein each mode comprises instructions to direct the system to perform one or more novel features of the present invention. The software according to the present invention may preferably be operated from a personal computer connected to SHCU 14 via a direct, hardwire connection or via a communications network, such that remote operation of the system is possible. It will be understood to one of ordinary skill in the art that the software and/or operating system may be designed differently while still achieving the same purposes. In all modes, the software can create, manipulate, and display to the user via a video monitor accurate, real-time three-dimensional images of the human body, which images can be zoomed, enlarged, rotated, animated, marked, segmented and referenced by the operator via the system's data input device(s). As described above, in various embodiments of the present invention the software and SHCU 14 can partially or fully control various attached components, probes, needles or devices to automate various functions of such components, probes, needles or devices, or facilitate robotic or remote control thereof.

The SHCU is also preferably operatively connected to one or more external imaging sources such as an magnetic resonance imaging (MRI), ultrasound (US), electrical impedance tomography (EIT), or any other imaging device known in the art and capable of creating images of the human body.

Treatment Protocols

In each of the disclosed embodiments, the method begins by locating a tumor using magnetic resonance imaging (MRI), ultrasound (US), electrical impedance tomography (EIT), or any other imaging device known in the art and capable of creating images of the human body. Location may be done manually using one of these known imaging devices or methods, and may or may not then be loaded into treatment software such as that run by the SHCU as described herein.

In certain embodiments, the treatment protocol may begin with the creation of a one or more 3D Fused Images Using of the patient's body in the region of the detected cancer, suspected neoplasia, or unwanted tissue inputs using magnetic resonance imaging (MRI), ultrasound (US), electrical impedance tomography (EIT), or any other imaging device known in the art and capable of creating images of the human body. In one embodiment, the SHCU may direct the creation of such an image via operative connection to one or more external sources including but not limited to imaging of the lumen of the patient's bodily structure. The 3D Fused Images provide a 3D map of the selected treatment area within the patient's body over which locational data obtained from the one or more probes or needles according to the present invention may be overlaid to allow the operator to monitor the treatment in real-time against a visual of the actual treatment area. Preferably, after the creation of a 3D Fused Image, a biopsy of the imaged area is taken (either immediately or at the convenience of the physician/patient) or the suspicion of tumor is confirmed by typical imaging characteristics.

In a first embodiment, a 3D Fused Image would be created from one or more MRI and ultrasound image(s) of the same area of the patient's body. An MRI image used for this purpose may comprise a multi-parametric magnetic resonance image created using, i.e., a 3.0 Telsa MRI scanner (such as Achieva, manufactured by Philips Healthcare) with a 16-channel cardiac surface coil (such as a SENSE coil, manufactured by Philips Healthcare) placed on the patient so as to support imaging of the area of concern in the patient. For example, for the treatment of prostate cancer, the surface coil may be placed over the pelvis of the patient with an endorectal coil (such as the BPX-30, manufactured by Medrad). For the treatment of sarcoma, MRI sequences obtained by this method preferably include: a tri-planar T2-weighted image, axial diffusion weighted imaging with apparent diffusion coefficient (ADC) mapping, 3-dimensional point resolved spatially localized spectroscopy, and an axial dynamic contrast enhanced MRI. An ultrasound image used for this purpose may be one or more 2D images obtained from one the use of equipment known in the art, including but not limited to: a standard biplane transrectal ultrasound probe (such as the Hitachi EUB 350), a standard biplane ultrasound transducer (such as the Hi Vision Preirus by Hitachi Aloka Medical America, Inc.), an endoscopic transducer such as an Olympus Curved Linear Array (GF-UC140P-AL5) connected to a ProSound F75 premium Hitachi Aloka, Ltd. ultrasound platform. The ultrasound image may be formed by, i.e., placing an EM field generator (such as that manufactured by Northern Digital Inc.) on the patient, which allows for real-time tracking of a custom ultrasound probe embedded with a passive EM tracking sensor (such as that manufactured by Traxtal, Inc.).

In some embodiments, the US and guidance can be carried out with the commercially available EPIQ 7 GI Ultrasound System, such as in the treatment of sarcoma or soft tissue tumors.

In one embodiment, the 3D fused image is then formed by the software according to the present invention by encoding the ultrasound data using position encoded data correlated to the resultant image by its fixed position to the US probe and/or transducer by the US scanning device. In an alternative embodiment, specifically for the treatment of prostate cancer, the 3D fused image is formed by encoding the ultrasound data using a position encoded prostate ultrasound stepping device (such as that manufactured by Civco Inc) and then overlaying a virtual brachytherapy grid over the 3D ultrasound fused MRI image. A brachytherapy grid is positionally correlated to the resultant image by its fixed position to the US probe by the US stepping device. Thus, in some embodiments, biopsy needle 200 does not need a locational sensor 26 because the positional guidance is provided by the brachytherapy grid. The software according to the present invention also records of the position of any obtained biopsy for later use in guiding therapy.

This protocol thus generates a baseline, diagnostic 3D Fused Image and optionally displays the diagnostic 3D Fused Image to the operator in real time via the SHCU video monitor. Preferably, the system may request and/or receive additional 3D ultrasound images of the treatment area during treatment and fuse those subsequent images with the baseline 3D Fused Image for display to the operator.

As an alternate means of creating the 3D Fused Image, a 2-dimensional sweep of the treatment area is performed in the axial plane to render a three-dimensional ultrasound image that is then registered and fused to a pre-biopsy MRI using landmarks common to both the ultrasound image and MRI image such as, for the treatment of prostate cancer, the capsular margins of the prostate and urethra. Where prostate cancer is the target, the sweep may be performed by a transrectal ultrasonography (TRUS) device. Lesions suspicious for cancer identified on MRI may be semi-automatically superimposed on the real-time US (or TRUS) image. A biopsy device (such as that manufactured by Bard, Inc.) and embedded with a passive EM tracking device, as previously described, can then be tracked in relation to the position any areas of concern and thus a biopsy performed or, in alternative embodiments, an intraluminal biopsy taken using a biopsy device (such as an Olympus EZ Shot 2 Aspiration Needle) placed through the catheter of the EMB probe 20.

In yet another embodiment, and specifically for the treatment of prostate cancer, the 3D Fused Image may be created by placing the patient in the dorsal lithotomy position, placing a biopsy grid on the perineum, inserting a TRUS probe into the rectum and placing the transducer in the proper position prior to 3D data acquisition at the lateral margin of the prostate. The operator then activates the ultrasound probe to capture multiple images. The computer then reconstructs a 3D image of the prostate by displaying the image in a multi-planer reformation (MPR) mode and displays grid lines through the 3D volume that correspond to the holes in the grid on the patient's perineum. At this point, the reconstructed MRI data can be fused to the ultrasound date using the previously described methods. Such a system was described in Onik G M, Downey D B, Fenster A, Sonographically Monitoring Cryosurgery In A Prostate Phantom, Journal of Ultrasound 16:267-270 (1996), which disclosure is incorporated herein in its entirety.

The 3D Fused Image as created by any one of the above methods may then be stored in the non-transitive memory of the SHCU, which may employ additional software to locate and electronically tag within the 3D Fused Image specific areas in the treatment area or its vicinity, including sensitive or critical structures and areas that require anesthesia such as, for example, the Neurovascular Bundles (for the treatment of prostate cancer), i.e. to enable the guidance of standard or trackable anesthesia needles to those locations. The SHCU then displays the 3D Fused Image to the operator alone or overlaid with locational data from each of the additional devices described herein where available. The 3D Fused Image may be presented in real time in sector view, or the software may be programmed to provide other views based on design preference.

As described above, the software may then direct the operator and/or a robotic arm to take a biopsy of the identified area of cancerous tissue or in a specific location of concern based on an analysis of the imaging data and record the results of same, which biopsy may be tracked in real time. Analysis of the biopsy tissue, which may be done by the system or a physician/technician, will indicate whether the biopsied tissue is cancerous. Thus, a 3D map of cancerous tissue in the area of concern within the patient's body may be created in this way. The software may employ an algorithm to determine where individual biopsies should be taken based on optimal spacing between same or based on the location of other biopsies that revealed cancerous tissue to ensure that all areas of cancerous tissue in the region have been located and indexed against the 3D Fused Image.

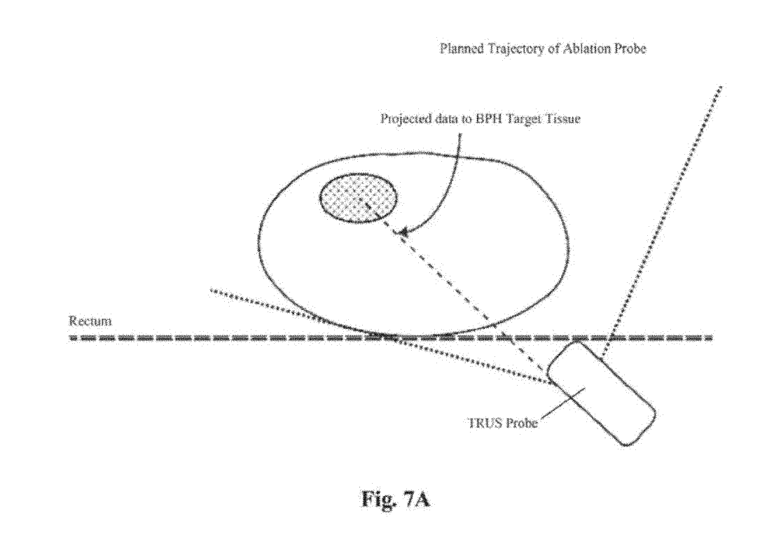

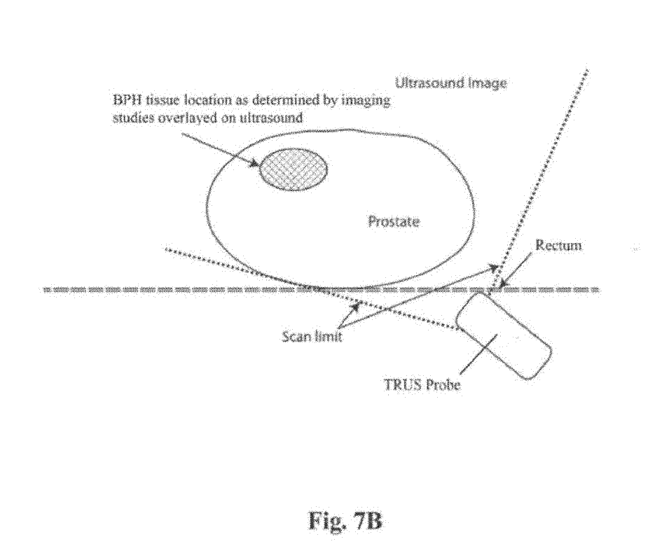

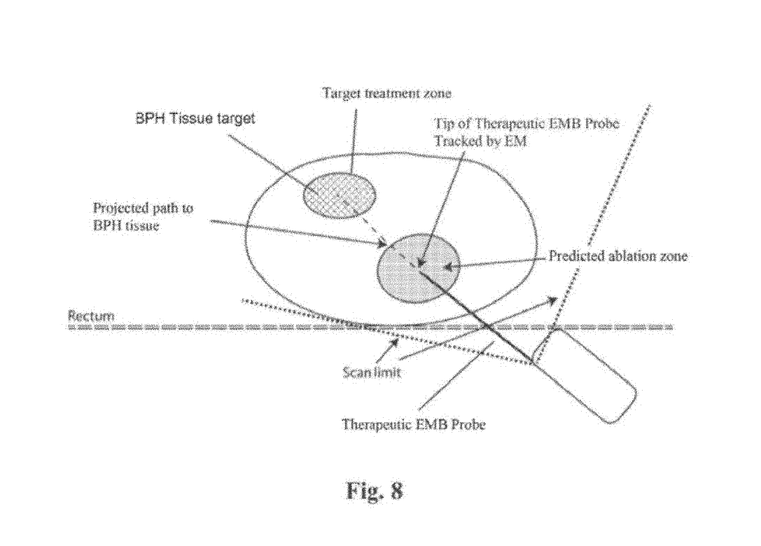

Using the biopsy result data in conjunction with the 3D Fused Image, the software can create a “3D Mapped Biopsy Fused Image”, which can be used as the basis for planning an office based or in-patient treatment procedure for the patient (see FIGS. 7A-7B). The SHCU also preferably stores the biopsy sample information indexed to sample location, orientation and number, which information can be provided to a pathologist or other treatment provider via a communications network to be displayed on his or her remote workstation, allowing the other treatment provider to interact with and record pathological findings about each sample in real time.

In embodiments utilizing one or more 3D Fused Images of the planned treatment area and/or biopsies of the affected area, the SHCU may display to the operator via a video terminal the precise location(s) of one or more areas in the treatment area, or its vicinity, which require therapy, via annotations or markers on the 3D Fused Image(s): this area requiring therapy is termed the Target Treatment Zone. This information is then used by the system or by a physician to determine optimal placement of the various probes used in the inventive methods. In certain embodiments, the 3D Fused Image should also contain indicia to mark Neurovascular Bundles (NVB), where present, or other anesthesia targets designated by the physician, the location of which will be used to calculate a path for placement of one or more anesthesia needles, where used, for delivery of local anesthesia to the treatment area. If necessary due to changes in gland, tumor or tissue size, the geographic location of each marker can be revised and repositioned, and the 3D Fused Image updated in real time by the software, using 3D ultrasound data as described above. The system may employ an algorithm for detecting changes in gland, tumor or tissue size and requesting additional ultrasound scans, may request ultrasound scans on a regular basis, or the like. In certain embodiments, the Target Treatment Zone encompasses less than the entire cancerous mass, so as to expose tumor antigens to the patient's immune system without destroying the entire tumor and/or the surrounding fluid pathways—the lymphatic and vascular systems—such that the immune response in the patient in response to this inventive treatment is amplified.

In a preferred embodiment, the software may provide one or more “virtual” EMB or CRYO/EMB treatment probes 20 (of the various types described above) which may be overlaid onto the 3D Fused Image by the software or by the treatment provider to determine the extent of target cell destruction that would be accomplished with each configuration. Where a non-catheter-type probe is used, the virtual probes also define a path to the target point by extending a line or path from the target point to a second point defining the entry point on the skin surface of the patient for insertion of the real EMB treatment probe. Preferably, the software is configured to test several possible probe 20 placements and calculate the probable results of treatment to the affected area via such a probe 20 (the Predicted Treatment Zone) placement using a database of known outcomes from various EMB or CRYO/EMB treatment protocols or by utilizing an algorithm which receives as inputs various treatment parameters such as pulse number, amplitude, pulse width and frequency. By comparing the outcomes of these possible probe locations to the tumor volume as indicated by the 3D Fused Image and/or the 3D Mapped Biopsy Fused Image, the system may determine the optimal probe 20 placement. Alternatively, the system may be configured to receive inputs from a physician to allow him or her to manually arrange and adjust the virtual EMB treatment probes to adequately cover the treatment area and volume based on his or her expertise. The system may utilize virtual anesthesia needles or any one or more of the other probes disclosed herein in the same way to plan treatment.

In certain embodiments, when the physician is satisfied with the Predicted Treatment Zone coverage shown on the Target Treatment Zone based on the placement and configuration of the virtual EMB or CRYO/EMB treatment probes and the virtual anesthesia needles or other ancillary probes, as determined by the system of by the physician himself, the physician “confirms” in the system (i.e. “locks in”) the three-dimensional placement and energy/medication delivery configuration of the grouping of virtual probe(s) and needle(s), and the system registers the position of each as an actual software target to be overlaid on the 3D Fused Image and used by the system for guiding the insertion or placement of the real probe(s) and needle(s) according to the present invention (which may be done automatically by the system via robotic arms or by the physician by tracking his or her progress on the 3D Fused Image).

An important step of the method according to a preferred embodiment is the injection of a immunostimulatory drug formulation after EMB or CRYO/EMB treatment to enhance the immunologic response of the patient to treatment. In this regard, it is important that the injection needle is properly positioned in the target tissue such that injections of therapeutic agents saturate the tumor tissue and force the mixture of immunostimulatory drugs and cellular antigens thus exposed by EMB or CRYO/EMB treatment into the interstitial fluid outside the target tissue. This process of “pushing” this mixture of immunostimulatory drugs and cellular antigens created by EMB or CRYO/EMB treatment forces these constituents to drain to the local draining lymph nodes of the patient where antigen presentation and T cell activation take place, thus enhancing the patient's own immunologic response to the EMB or CRYO/EMB treatment. Thus, a critical aspect of the treatment described herein is that the injection needle is not positioned in a blood vessel, because if it was, any injections of therapeutic agents would be carried away by the vasculature, and not saturate the tumor tissue as described.

Notwithstanding the above, in certain embodiments the treatment protocol(s) described herein may be accompanied by one or more pre-treatments of an immune-stimulant such as a cytokine GM-CSF. For example, such an immune-stimulatory drug could be delivered subcutaneously as a daily injection beginning one week prior to the planned treatment date using one or more of the herein-described protocols. However, single doses of an immune-response enhancing drug could be administered several weeks or several days in advance of the planned treatment, or a course of multiple treatments starting within the same range prior to treatment, based on the specific location and type of tumor and tissue to be treated.

In preferred embodiments, the same needle tract used to deliver EMB or CRYO/EMB treatment is used for injection of the immunostimulatory drug formulation. This may be accomplished using one of the herein-described treatment probes that incorporates an integral injection means, or may be done by removing a previously-placed treatment probe and inserting an appropriate injection means either through a lumen which remains in place throughout treatment or through the same injection tract as used by prior treatment probe(s). Thus, in these embodiments, placement of the EMB or CRYO/EMB probe outside of a blood vessel is crucial. In certain embodiments, in addition to or as an alternative to the utilization of a 3D Fused Image and/or virtual treatment probes, contrast material may be used upon insertion of the treatment probe ensure that the placement of the needle is properly within the target (tumor) tissue and not in a blood vessel.

If necessary, EMB treatment, as described in further detail below, may be carried out immediately after a biopsy of the patient is performed. Alternately, EMB treatment may take place days or even weeks after one or more biopsies are performed. In the latter case, the steps described with respect to the Planning Mode of the SHCU and related software, described above, may be undertaken by the software/physician at any point between biopsy(s) and treatment.

In embodiments comprising guidance of the treatment protocol via the SHCU, the software displays, via the SHCU video monitor, the previously confirmed and “locked in” Target Treatment Zone, Predicted Treatment Zone and 3D Mapped Biopsy Fused Image, with the location and configuration of all previously confirmed virtual probes/needles and their calculated insertion points, angular 3D geometry, and insertion depths or placement when inserted intraluminally, which can be updated as needed at time of treatment to reflect any required changes as described above.

Optionally, using the planned locations and targets established for the delivery of anesthesia, and the displayed insertions paths, the software then guides the physician (or robotic arm) in real time to place one or more anesthesia needles and then to deliver the appropriate amount of anesthesia to the targeted locations (i.e., in the vicinity of the Neurovascular Bundles). Deviations from the insertion path previously determined by the system in relation to the virtual or placement location of the needles/probes may be highlighted by the software in real time so as to allow correction of targeting at the earliest possible time in the process. This same process allows the planning and placement of local anesthesia needles as previously described. In some embodiments, the system may employ an algorithm to calculate the required amount of anesthesia based on inputs such as the mass of the tissue to be treated and individual characteristics of the patient which may be inputted to the system manually by the operator or obtained from a central patient database via a communications network, etc.

Once anesthesia, if used, has been administered, the system displays the Predicted Treatment Zone and the boundaries thereof as an overlay on the 3D Fused Image including the Target Treatment Zone and 3D Mapped Biopsy Fused Image and directs the physician (or robotic arm) as to the placement of each EMB or CRYO/EMB treatment probe 20. The Predicted Treatment Zone may be updated and displayed in real time as the physician positions each probe 20 to give graphic verification of the boundaries of the Target Treatment Zone, allowing the physician to adjust and readjust the positioning of the Therapeutic EMB Probes, sheaths, electrode exposure and other treatment parameters (which in turn are used to update the Predicted Treatment Zone). When the physician (or, in the case of a fully automated system, the software) is confident of accurate placement of the probes, he or she may provide such an input to the system, which then directs the administration of EMB and/or CRYO/EMB treatment as herein described.

In a preferred embodiment, as described above, a guidance needle is first positioned in the target tissue and the physician, or in the case of a guided, semi-automated or automated protocol, the system, verifies that the needle is properly positioned and not residing within a blood vessel. Verification may be by guided imagery as described above, by the injection of contrast media, and/or by the use of a spot biopsy at the injection site.

Next, according to this preferred embodiment, the guidance needle is removed and an EMB and/or CRYO/EMB probe, as described herein, is inserted through the same needle tract. An EMB and/or CRYO/EMB treatment protocol is then administered, preferably under the direction of the SHCU which is operably connected to the EMB and/or CRYO/EMB probe, the cryogenic freezing unit/cryomachine and the EMB pulse generator as described above.

The pulse amplitude 30, frequency 31, polarity and shape provided by the EMB pulse generator 16, as well as the number of pulses 32 to be applied in the treatment series or pulse train, the duration of each pulse 32, and the inter pulse burst delay 33 are designed to expose tumor antigens without damage to same, and without damage to the critical fluid pathways that the immune system requires to function effectively. This process is preferably controlled by the SHCU. Although only two are depicted in FIG. 10 due to space constraints, in one embodiment EMB treatment is preferably performed by application of a series of not less than 100 electric pulses 32 in a pulse train so as to impart the energy necessary on the target tissue 2 without developing thermal issues in any clinically significant way. The width of each individual pulse 32 is preferably from 100 to 1000 μs with an inter pulse burst interval 33 during which no voltage is applied in order to facilitate heat dissipation and avoid thermal effects.

As described herein, pre-treatment of the target tissue with a cryoablation protocol reduces the ultimate duration of the EMB treatment by “hardening” the cellular membranes of the target cells, making them more prone to breakage during the rapid flexing motion to which they are subjected during the EMB treatment protocol. Thus, according to a preferred embodiment, between 1 and 10 cryoablative freeze cycles of between 30 to 240 seconds each are performed prior to the application of electric current as described with reference to the EMB treatment protocol. In a most preferred embodiment, the cryoablative “pre-cycle” includes 1 freeze cycle of between 90 and 120 seconds. Because the cryoablative pre-cycle pre-stresses the cell membrane, as described herein, the following EMB treatment protocol requires less energy to achieve the same result as without said cryoablative pre-cycle. Therefore, in this most preferred embodiment, a cryoablative “pre-cycle” of 1 freeze cycle lasting between 90 and 120 seconds is followed by an EMB treatment cycle of between 1 and 100 pulses, with additional characteristics of said EMB treatment cycle within the parameters described above.

Various types of tissue respond differently to both cryoablative techniques and EMB treatment. Thus in preferred embodiments, the system automatically adjusts (or suggests appropriate parameters to the clinician) the optimized treatment based on the type of tissue comprising the target. For example, in certain types of tissue, the system may perform two freezing cycles, either before EMB treatment is applied, or after one or more EMB cycles, or the like.

The relationship between the duration of each pulse 32 and the frequency 31 (period) determines the number of instantaneous charge reversals experienced by the cell membrane during each pulse 32. The duration of each inter pulse burst interval 33 is determined by the controller 14 based on thermal considerations. In an alternate embodiment the system is further provided with a temperature probe 22 inserted proximal to the target tissue 2 to provide a localized temperature reading at the treatment site to the SHCU 14. The temperature probe 22 may be a separate, needle type probe having a thermocouple tip, or may be integrally formed with or deployed from one or more of the needle electrodes, or the Therapeutic EMB Probes. The system may further employ an algorithm to determine proper placement of this probe for accurate readings from same. With temperature feedback in real time, the system can modulate treatment parameters to eliminate thermal effects as desired by comparing the observed temperature with various temperature set points stored in memory. More specifically, the system can shorten or increase the duration of each pulse 32 to maintain a set temperature at the treatment site to, for example, create a heating (high temp) for the needle tract to prevent bleeding or to limit heating (low temp) to prevent any coagulative necrosis. The duration of the inter pulse burst interval can be modulated in the same manner in order to eliminate the need to stop treatment and maximizing the deposition of energy to accomplish EMB. Pulse amplitude 30 and total number of pulses in the pulse train may also be modulated for the same purpose and result. The EMB or CRYO/EMB protocol is thus preferably optimized to create a spherical treatment zone of 1.5 cm or less in diameter.

In yet another embodiment, the SHCU may monitor or determine current flow through the tissue during treatment for the purpose of avoiding overheating while yet permitting treatment to continue by reducing the applied voltage. Reduction in tissue impedance during treatment due to charge buildup and membrane rupture can cause increased current flow which engenders additional heating at the treatment site. With reference to FIG. 6, prior treatment methods have suffered from a need to cease treatment when the current exceeds a maximum allowable such that treatment goals are not met. As with direct temperature monitoring, the present invention can avoid the need to stop treatment by reducing the applied voltage and thus current through the tissue to control and prevent undesirable clinically significant thermal effects. Modulation of pulse duration and pulse burst interval duration may also be employed by the controller 14 for this purpose as described.

In a preferred embodiment, during treatment, the software captures all of the treatment parameters, all of the tracking data and representational data in the Predicted Treatment Zone, the Target Treatment Zone and in the 3D Mapped Biopsy Fused Image as updated in real time to the moment of therapeutic trigger. Based on the data received by the system during treatment, the treatment protocol may be adjusted or repeated as necessary.

The software may also store, transmit and/or forwarding treatment data to a central database located on premises in the physician's office and/or externally via a communications network so as to facilitate the permanent archiving and retrieval of all procedure related data. This will facilitate the use and review of treatment data, including for diagnostic purposes and pathology related issues, for treatment review purposes and other proper legal purposes including regulatory review.

The software may also transmit treatment data in real time to a remote proctor/trainer who can interact in real time with the treating physician and all of the images displayed on the screen, so as to insure a safe learning experience for an inexperienced treating physician, and so as to archive data useful to the training process and so as to provide system generated guidance for the treating physician. In another embodiment, the remote proctor can control robotically all functions of the system.

In preferred embodiments, once EMB or CRYO/EMB treatment has been performed and tumor antigens have been exposed, the CRYO/EMB needle probe is exchanged via the same needle tract (or lumen) with an injection needle. Alternatively, the entire treatment protocol described herein is performed via one of the treatment probes described above which incorporate an integral means for injection of fluids such as an immunologic response enhancing drug. With the injection needle tip is residing in the center of the antigen exposure zone, one or more injections of an immune response enhancing drug formulation are performed.

The drug components of the drug formulation are chosen to achieve two outcomes: 1. To mitigate the tumor's ability to locally turn off or down regulate antitumor immune responses, and 2. To stimulate the immune system to interact with the tumor antigens and form an autologous therapeutic tumor vaccine. The drug formulation in the preferred embodiment is comprised of either sequential injections of a CTLA-4 checkpoint inhibitor (2 ml), followed by a PD-1 checkpoint inhibitor (2 ml) followed by a cytokine GM-CSF immune-stimulant (1 ml). Another embodiment involves injecting a formulation of the immunotherapeutics mentioned simultaneously, either via 3 syringes actuated simultaneously, or via a combination formulation comprised of all three drugs. In other embodiments, the injection can simply comprise one or more immune checkpoint inhibitors, including one or more of those described herein or developed in the future. Alternatives to the dosage and volume of the applied drug may be contemplated based on design choice based on the preferred ranges of same indicated for each specific drug to be used, and such revisions are within the scope of the present invention.

The injection is intended to physically force the newly exposed tumor cell contents and membrane fragments into the tissue drainage system. The primary tissue drainage system for a solid tumor is the lymphatic system, and this is where the newly exposed cellular contents, which include the tumor antigens, are primarily forced. The lymphatic system is also where the immune system is most highly concentrated and where it works most effectively.

One important feature of the drug formulation is that it should have appropriate viscosity to allow for the injectate to “push” the newly-exposed cellular contents, including the tumor antigens, into the tissue drainage system, especially the lymphatic system which is where the immune system resides and works most efficiently. The formulation according to the proposed invention may be provided in an aqueous suspension, or in a more viscous suspension of the immunotherapeutics and immunostimulants, such as a hydrogel. If a higher viscosity formulation is utilized, the flow rates should still be titered to ensure that the injectate is not escaping along the needle tract, and is remaining in the tumor tissue so that it forces the newly exposed and liberated tumor cell contents into the lymphatic drainage system.

For purposes of the present invention a low, medium or high viscosity solution may be used, depending on the target tissue and other treatment considerations. For example, for certain applications, a low viscosity s of 1 cP (centipoise, or the equivalent of 1 mPa·s milli-Pascal second) can be used for applications in which it is desired to allow the injectate to disperse into the treatment zone tissue efficiently. In other preferred embodiments, medium to high viscosity formulations having a viscosity range of 20-100 cP for medium viscosity, and up to 14,000 cP for high viscosity formulations, may be used. In these embodiments, medium to high viscosity formulations have the advantage that they will reside in the treatment zone because they are too viscous to flow away, and elute the applied drugs into the treatment zone in a controlled fashion. Since these higher viscosity formulations stay at the point at which they are injected for a considerably longer period than low viscosity injections will, they mechanically create a space for the injection which pushes the treatment zone tissue out of the way. This injection space creation provides feedback to the clinician that the drugs are residing in the correct position within the treatment zone. This also provides the ability to elute the drugs in the formulation into the surrounding tissue at a controlled rate that ranges between 2 minutes and 6 hours.

The injection into the solid mass of target tissue needs to be forceful enough to overcome the back pressure of the tissue it is being injected into, but not so forceful that it overcomes the seal between the needle shaft and the tumor tissue. If the injection rate is too forceful the injectate will flow out of the tissue along the needle tract and it will not force the newly exposed tumor antigens and other cellular contents into the lymphatic system. This effect is accomplished, in a most preferred embodiment, by an injection carried out at a constant volume, with an injection pressure that is dependent on the type of tissue into which the injectate will be applied. To achieve this goal, the injector coupled to the injection needle is calibrated for flow rate of 0.008 through 0.5 ml per minute, but most preferably approximately 0.2 ml per minute or lower based upon the makeup of the target tissue. This preferred flow rate may be increased or decreased based on imaging feedback to ensure that the injectate is not leaking back along the needle tract. Contrast media can be mixed with the injectate to aid in visualization of the any leakage around the needle tract and also to see the injectate flush out into the lymphatic system.

In the preferred embodiment, the volume of injectate is approximately 3 times the volume of the treatment zone. Thus, if the treatment zone is a sphere of 1.5 cm in diameter, equivalent to a volume of 1.77 ml, the volume of the injectate is approximately 5 ml.

In other embodiments, the volume, pressure, and viscosity of injectate are designed to fill the treatment area without significant additional volume. In such embodiments, the injectate and drug formulation will drain by diffusion to the patient's lymphatic system. For example, where the treatment tissue comprises brain tissue, a lower volume or pressure injection may be desired. Importantly, the device used to perform the injection should not utilize a pressure-dependent cutoff, but should be capable of applying consistent pressure, where needed, to achieve the treatment goals described herein. However, it will also be understood that, as mentioned above, in certain embodiments the injectate will be designed to over-fill the treatment area, and so larger injection volumes may be used depending on tissue type, treatment area and other design choice.

In other embodiments of the present invention, some or all of the treatment protocol may be completed by robotic arms, which may include a treatment probe guide which places the specially designed Therapeutic EMB Probe (or an ordinary treatment probe but with limitations imposed by its design), or other probe types described herein, in the correct trajectory or intraluminal location relative to the tumor. Robotic arms may also be used to hold the US transducer in place and rotate it to capture images for a 3D US reconstruction. Robotic arms can be attached to an anesthesia needle guide which places the anesthesia needle in the correct trajectory to the targeted anesthesia areas to guide the delivery of anesthesia by the physician.

In other embodiments, the robotic arm can hold the anesthesia needle itself or a trackable anesthesia needle (see FIG. 14) with sensor-transmitters and actuators built in, that can be tracked in real time, and that can feed data to the software to assure accurate placement thereof and enable the safe, accurate and effective delivery of anesthesia to the targeted anesthesia areas and other regions, and can directly insert the needle into the targeted areas of the Neurovascular Bundle and other regions using and reacting robotically to real time positioning data supported by the 3D Mapped Biopsy Fused Image and Predicted Treatment Zone data and thereby achieving full placement robotically, and upon activation of the flow actuators, the delivery of anesthesia as planned or confirmed by the physician.

In addition, the robotic arm can hold the Therapeutic EMB Probe itself and can directly insert the probe into the patient's tumor (or into an intraluminal location proximate the tumor) using and reacting robotically to real time positioning data supported by the 3D Mapped Biopsy Fused Image and Predicted Treatment Zone data and thereby achieving full placement robotically.

Robotic components capable of being used for these purposes include the iSR'obot™ Mona Lisa robot, manufactured by Biobot Surgical Pte. Ltd. In such embodiments the Software supports industry standard robotic control and programming languages such as RAIL, AML, VAL, AL, RPL, PYRO, Robotic Toolbox for MATLAB and OPRoS as well as other robot manufacturer's proprietary languages.

In yet another embodiment, tissue characterization ability which is built into the EMB probe itself can identify the cancerous area and then allow direct destruction of the tumor in a one step procedure eliminating the need for the separate biopsy and pathological examination.

In yet another embodiment, the treatment is applied to a metastatic lesion in an organ other than the primary cancer organ, using all the capabilities of the system outlined above. In the treatment of a metastatic lesion, the lesion may further be directly injected with immune enhancing drugs to facilitate a tumor specific immune response.

In another embodiment, the disease type treated is a squamous cell carcinoma or basal cell carcinoma. In yet another embodiment, the skin lesion treated is a benign lesion such as a neurofibroma. In yet another embodiment, the skin lesion treated is a lipoma located subcutaneously.

In other embodiments, the system as described above is used to treat prostate neoplasia or BPH from an intraurethral location. In yet other embodiments, the system is used to treat esophageal carcinoma or Barret's esophagus.

In yet another embodiment, the system with the intraluminal probe is used inside the bile duct, pancreatic duct or bowel to treat pancreatic carcinoma. In another embodiment, the system using the intraluminal probe is used to treat bile duct carcinoma from an intraluminal location inside the bile duct.

The SHCU can fully support Interactive Automated Robotic Control through a proprietary process for image sub-segmentation of the treatment area and nearby anatomical structures for planning and performing robotically guided biopsy and therapeutic interventions in an office based or in-patient setting.

Sub-segmentation is the process of capturing and storing precise image detail of the location size and placement geometry of the described anatomical object so as to be able to define, track, manipulate and display the object and particularly its three-dimensional boundaries and accurate location in the body relative to the rest of the objects in the field and to the anatomical registration of the patient in the system so as to enable accurate three-dimensional targeting of the object or any part thereof, as well as the three-dimensional location of its boundaries in relation to the locations of all other sub segmented objects and computed software targets and needle and probe pathways. The software sub-segments out various critical substructures in or proximate to the treatment area, such as the neuro-vascular bundles, peripheral zone, ejaculatory ducts, urethra, rectum, and Denonvilliers Fascia in a systematic and programmatically supported and required fashion, which is purposefully designed to provide and enable the component capabilities of the software as described herein.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth herein.

We claim:

1. A method of ablating undesirable soft tissue in a living subject, comprising the steps of:
identifying a location of said soft tissue within said subject;
determining a position of at least one electrode relative to said soft tissue;
introducing at least one treatment probe to said position within said subject, said treatment probe comprising said at least one electrode and means for conveying a cooled gas, said at least one electrode electrically connected to a controller for controlling the delivery of electric pulses to said electrode, said controller comprising an electric pulse generator;
applying to said soft tissue, via said at least one treatment probe, at least one cryoablation cycle;
applying to said soft tissue an electric field, said electric field applied to said soft tissue by delivering from said pulse generator to said at least one electrode at least one bi-polar pulse train, said bi-polar pulse train comprising at least two bi-polar electric pulses, each said bi-polar electric pulse in said bi-polar pulse train being separated by an inter pulse burst interval during which no voltage is applied to said at least one electrode;
wherein a voltage of each of said bi-polar electric pulses is from 0.5 kV to 10 kV.

2. The method of claim 1 wherein a frequency of said electric field is from 14.2 kHz to less than 500 kHz.

3. The method of claim 1 wherein said frequency of said electric field is from 100 kHz to 450 kHz.

4. The method of claim 1 wherein said voltage over time of each of said bi-polar electric pulses traces a square waveform for a positive and negative component of a polarity oscillation.

5. The method of claim 1 wherein said voltage of each of said bi-polar electric pulses is characterized by waveforms with an instant charge reversal, between the positive and negative charge of each cycle.

6. The method of claim 1 wherein said at least one cryoablation cycle comprises between 1 and 10 cryoablative freeze cycles of between 30 to 240 seconds each.

7. The method of claim 6 wherein said at least one cryoablation cycle comprises a single cryoablation cycle lasting between 90 and 120 seconds.

8. The method of claim 7 wherein said at least two bi-polar electric pulses comprises between 2 and 100 pulses.

9. The method of claim 1 wherein the duration of each of said at least one bi-polar electric pulses is from 100-1000 μs.

10. The method of claim 1, further comprising injecting at least one immunologic response enhancing drug into said soft tissue.

11. A system for ablating undesirable soft tissue in a living subject, the system comprising:
at least one treatment probe comprising at least one electrode;
an electric pulse generator electrically connected to said treatment probe;
a cryomachine operatively connected to said at least one treatment probe.

12. The system of claim 11, wherein said at least one electrode comprises a first electrode disposed on a distal end of said at least one treatment probe, and further comprising an indifferent electrode located physically remotely from said first electrode,
wherein said first electrode and said indifferent electrode are both electrically connected to said electric pulse generator.

13. The system of claim 11, wherein said at least one treatment probe comprises:
a central portion comprising a central gas supply cannula operatively in fluid connection with said cryomachine;
a concentric portion electrically connected to said electric pulse generator;
a layer of thermal insulation surrounding said central portion; and
a layer of electrical insulation surrounding said concentric portion;
whereby said central portion and said concentric portion are repositionable relative to one another.

14. The system of claim 13, wherein the central portion is made of an electrically conductive material, and wherein said concentric portion further comprises electrical contacts to transmit electrical impulses from said electric pulse generator to said central portion.

15. The system of claim 11, further comprising means for injecting one or more fluids into tissue located at a distal end of said at least one treatment probe.

16. The system of claim 11, further comprising a software hardware control unit for controlling the delivery of electric pulses from said electric pulse generator to said at least one electrode, and for controlling the delivery of cooled gas from said cryomachine to a distal end of said at least one treatment probe.

17. The system of claim 12, wherein said at least one treatment probe comprises a central lumen sized to accommodate at least one standard gauge needle.
